SEQUENCE LISTING (5 sequences)

SEQ ID NO: 1 - protein, 52 aa, synthetic construct
EKQLRRVEPEHEDNTRVEARPRALLEAKTKELVEPTSKEAEEARQILAEQAA

SEQ ID NO: 2 - protein, 59 aa, synthetic construct
YENSEDVAVPSDSASTPDCEEQQEQGDTTLSDHDFHSGGTEQEGLPETEVAHQHETEEQ

SEQ ID NO: 3 - protein, 10 aa, synthetic construct
HPGSVNEFDF

SEQ ID NO: 4 - protein, 9 aa, synthetic construct
IPAAAGRFF

SEQ ID NO: 5 - protein, 9 aa, synthetic construct
SPMNGGYYM

(19) United States
(12) Patent Application Publication
(10) Pub. No.: US 2025/0067735 A1
(43) Pub. Date: Feb. 27, 2025

(54) TREATMENT AND DIAGNOSIS OF TOXOPLASMA-ASSOCIATED DISEASE

(71) Applicant: The Johns Hopkins University, Baltimore, MD (US)

(72) Inventors: Jianchun Xiao, Baltimore, MD (US); Raphael P. Viscidi, Baltimore, MD (US); Robert H. Yolken, Baltimore, MD (US)

(21) Appl. No.: 18/723,348

(22) PCT Filed: Dec. 23, 2022
(86) PCT No.: PCT/US22/82361

§ 371 Date: Jun. 21, 2024

Related U.S. Application Data
(60) Provisional application No. 63/293,231, filed on Dec. 23, 2021.

(51) Int. Cl. G01N 33/569; C07K 14/45

(52) U.S. Cl. CPC G01N 33/56905; C07K 14/45; G01N 2333/45; G01N 2469/20

(57) ABSTRACT

Methods of predicting risk of subsequent diseases following Toxoplasma infection in multiple at-risk populations including patients with ocular toxoplasmosis or congenital toxoplasmosis, transplant recipients and other immunosuppressed patients with prior exposure to toxoplasma. The methods include the use of synthetic modified Toxoplasma cyst peptides.

### CROSS-REFERENCES TO RELATED APPLICATIONS

This application claims the benefit of priority of U.S. Provisional Application No. 63/293,231 filed on Dec. 23, 2021, which is incorporated herein by reference in its entirety and for all purposes.

### BACKGROUND

Toxoplasmic encephalitis (TE) is a brain infection with high morbidity and mortality, which almost always represents reactivation of a previously acquired parasitic infection with Toxoplasma gondii [1]. Exposure to Toxoplasma is common in the general population, but the incidence of TE is low and the risk factors for it have been difficult to characterize. The most important risk factor for TE is immunosuppression, although some Toxoplasma-infected AIDS patients never developed TE despite being severely immunocompromised [1]. Other factors such as host genetics, differences in virulence of Toxoplasma strains, and the residual burden of the parasite in the brain may also contribute to the risk of disease [2-4]. Elevated levels of antibody to the whole Toxoplasma organism have also been associated with increased risk for TE [5-7]. The clinical signs and symptoms of TE are nonspecific; therefore, definitive diagnosis requires either a brain biopsy or unequivocal response to Toxoplasma specific therapy. The poor predictive value of known risk factors for TE among Toxoplasma-infected individuals and the challenges of making a definitive diagnosis argue for the need to develop robust prediagnostic markers for TE to better manage patients at risk.

### SUMMARY

Provided herein are compositions and methods of use in diagnosing and predicting risk of subsequent diseases following Toxoplasma infection in multiple at-risk populations.

Accordingly, in certain aspects, a method of diagnosing Toxoplasma-associated disease in a subject comprises obtaining a biological sample from the subject; culturing the biological sample with a modified Toxoplasma cyst peptide; measuring the amount of antibodies binding to the modified Toxoplasma cyst peptide; and diagnosing Toxoplasma-associated disease in the subject. In certain embodiments, the modified Toxoplasma cyst peptide comprises a Toxoplasma matrix antigen 1 (MAG1) peptide comprising one or more amino acid deletions, amino acid substitutions, amino acid insertions or combinations thereof.

In certain embodiments, the modified Toxoplasma cyst peptide comprises an amino acid sequence having at least a 50% sequence identity to EKQLRRVEPEHEDNTRVEAR/P/RALLEAKTKELVEPTSKEAEEARQILAEQAA (SEQ ID NO: 1). In certain embodiments, the modified Toxoplasma cyst peptide comprises an amino acid sequence having at least a 75% sequence identity to EKQLRRVEPEHEDNTRVEAR/P/RALLEAKTKELVEPTSKEAEEARQILAEQAA (SEQ ID NO: 1). In certain embodiments, the modified Toxoplasma cyst peptide comprises an amino acid sequence having at least a 95% sequence identity to EKQLRRVEPEHEDNTRVEAR/P/RALLEAKTKELVEPTSKEAEEARQILAEQAA (SEQ ID NO: 1). In certain embodiments, the modified Toxoplasma cyst peptide comprises an amino acid sequence of EKQLRRVEPEHEDNTRVEAR/P/RALLEAKTKELVEPTSKEAEEARQILAEQAA (SEQ ID NO: 1). In certain embodiments, the modified Toxoplasma cyst peptide comprises an amino acid sequence having at least a 50% sequence identity to YENSEDVAVPSDSASTP/DCEEQQEQGDTTLSDHDFHSGGTEQEGLPETEVAHQHET EEQ (SEQ ID NO: 2). In certain embodiments, the modified Toxoplasma cyst peptide comprises an amino acid sequence having at least a 75% sequence identity to YENSEDVAVPSDSASTP/DCEEQQEQGDTTLSDHDFHSGGTEQEGLPETEVAHQHET EEQ (SEQ ID NO: 2). In certain embodiments, the modified Toxoplasma cyst peptide comprises an amino acid sequence having at least a 95% sequence identity to YENSEDVAVPSDSASTP/DCEEQQEQGDTTLSDHDFHSGGTEQEGLPETEVAHQHET EEQ (SEQ ID NO: 2). In certain embodiments, the modified Toxoplasma cyst peptide comprises an amino acid sequence of YENSEDVAVPSDSASTP/DCEEQQEQGDTTLSDHDFHSGGTEQEGLPETEVAHQHET EEQ (SEQ ID NO: 2). In certain embodiments, the Toxoplasma-associated disease is toxoplasmic encephalitis or Toxoplasma-associated neurological disorders comprising neuropsychiatric and degenerative diseases. In certain embodiments, the Toxoplasma-associated disease is toxoplasmic retinitis. In certain embodiments, the Toxoplasma-associated disease is toxoplasmic encephalitis. In certain embodiments, the subject is treated with an anti-Toxoplasma agent or other therapeutic agents. In certain embodiments, the therapeutic agents comprise: Trimethoprim-Sulfamethoxazole (TMP-SMX), pyrimethamine; Sulfadiazine in combination with pyrimethamine, Clindamycin, Minocycline, Folinic acid, antibiotics pain relievers, anti-pyretic agents, and the like.

In certain embodiments, the subject is immunocompromised.

In another aspect, a kit comprises a modified Toxoplasma cyst peptide. In certain embodiments, the modified Toxoplasma cyst peptide comprises an amino acid sequence having at least a 50% sequence identity to EKQLRRVEPEHEDNTRVEAR/P/RALLEAKTKELVEPTSKEAEEARQILAEQAA (SEQ ID NO: 1). In certain embodiments, the modified Toxoplasma cyst peptide comprises an amino acid sequence having at least a 75% sequence identity to EKQLRRVEPEHEDNTRVEAR/P/RALLEAKTKELVEPTSKEAEEARQILAEQAA (SEQ ID NO: 1). In certain embodiments, the modified Toxoplasma cyst peptide comprises an amino acid sequence having at least a 95% sequence identity to EKQLRRVEPEHEDNTRVEAR/P/RALLEAKTKEL VEPTSKEAEEARQILAEQAA (SEQ ID NO: 1). In certain embodiments, the modified Toxoplasma cyst peptide comprises an amino acid sequence of EKQLRRVEPEHEDNTRVEAR/P/RALLEAKTKELVEPTSKEAEEARQILAEQAA (SEQ ID NO: 1). In certain embodiments, the modified Toxoplasma cyst peptide comprises an amino acid sequence having at least a 50% sequence identity to YENSEDVAVPSDSASTP/DCEEQQEQGDTTLSDHDFHSGGTEQEGLPETEVAHQHET EEQ (SEQ ID NO: 2). In certain embodiments, the modified Toxoplasma cyst peptide comprises an amino acid sequence having at least a 75% sequence identity to YENSEDVAVPSDSASTP/DCEEQQEQGDTTLSDHDFHSGGTEQEGLPETEVAHQHET EEQ (SEQ ID NO: 2). In certain embodiments, the modified Toxoplasma cyst peptide comprises an amino acid sequence having at least a 95% sequence identity to YENSEDVAVPSDSASTP/DCEEQQEQGDTTLSDHDFHSGGTEQEGLPETEVAHQHET EEQ (SEQ ID NO: 2). In certain embodiments, the modified Toxoplasma cyst peptide comprises an amino acid sequence of YENSEDVAVPSDSASTP/DCEEQQEQGDTTLSDHDFHSGGTEQEGLPETEVAHQHET EEQ (SEQ ID NO: 2). In certain embodiments, the modified Toxoplasma cyst peptides are immobilized to a solid medium. In certain embodiments, the modified Toxoplasma cyst peptides are in solution. In certain embodiments, the modified Toxoplasma cyst peptides are in a semi-solid medium. In certain embodiments, the modified Toxoplasma cyst peptides are lyophilized. In certain embodiments, the kit further comprising one or more secondary antibodies or antigen binding fragments thereof, for detecting antibodies bound to the modified Toxoplasma cyst peptides. In certain embodiments, the one or more secondary antibodies or antigen binding fragments thereof, comprise a detectable label. In certain embodiments, the reporter molecule or detectable label comprises fluorophores, radiochemical, luminescent compounds, electron-dense reagents, enzymes, biotin, radioactive compounds, non-radioactive compounds, digoxigenin, or haptens. In certain embodiments, the kit optionally comprises a capture antibody which specifically binds to the one or more modified Toxoplasma cyst peptides. In certain embodiments, the capture antibody binds to an epitope distinct to that of an antibody present in a biological sample of a subject infected with a Toxoplasma parasite. In certain embodiments, Toxoplasma parasite is Toxoplasma gondii.

In certain aspects a synthetic peptide comprises an amino acid sequence having at least a 50% sequence identity to EKQLRRVEPEHEDNTRVEAR/P/RALLEAKTKELVEPTSKEAEEARQILAEQAA (SEQ ID NO: 1). In certain embodiments, the peptide comprises an amino acid sequence having at least a 75% sequence identity to EKQLRRVEPEHEDNTRVEAR/P/RALLEAKTKELVEPTSKEAEEARQILAEQAA (SEQ ID NO: 1). In certain embodiments, the peptide comprises an amino acid sequence having at least an 80%, 85%, 90%, 95%, 98% or 99% sequence identity to EKQLRRVEPEHEDNTRVEAR/P/RALLEAKTKELVEPTSKEAEEARQILAEQAA (SEQ ID NO: 1). In certain embodiments, the peptide comprises an amino acid sequence of

(SEQ ID NO: 1)
EKQLRRVEPEHEDNTRVEAR/P/RALLEAKTKELVEPTSKEAEEAR
QILAEQAA.

In certain aspects, a synthetic peptide comprises an amino acid sequence having at least a 50% sequence identity to YENSEDVAVPSDSASTP/DCEEQQEQGDTTLSDHDFHSGGTEQEGLPETEVAHQHET EEQ (SEQ ID NO: 2). In certain embodiments, the peptide comprises an amino acid sequence having at least a 75% sequence identity to YENSEDVAVPSDSASTP/DCEEQQEQGDTTLSDHDFHSGGTEQEGLPETEVAHQHET EEQ (SEQ ID NO: 2). In certain embodiments, the peptide comprises an amino acid sequence having at least an 80%, 85%, 90%, 95%, 98% or 99% sequence identity to YENSEDVAVPSDSASTP/DCEEQQEQGDTTLSDHDFHSGGTEQEGLPETEVAHQHET EEQ (SEQ ID NO: 2). In certain embodiments, the peptide comprises an amino acid sequence of

(SEQ ID NO: 2)
YENSEDVAVPSDSASTP/DCEEQQEQGDTTLSDHDFHSGGTEQEGL
PETEVAHQHETEEQ.

### Definitions

The terminology used herein is for the purpose of describing particular embodiments only and is not intended to be limiting of the invention. Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the term “about” in the context of a numerical value or range means±10% of the numerical value or range recited or claimed, unless the context requires a more limited range.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The term “amino acid” includes naturally-occurring α-amino acids and their stereoisomers, as well as unnatural (non-naturally occurring) amino acids and their stereoisomers. “Stereoisomers” of amino acids refers to mirror image isomers of the amino acids, such as L-amino acids or D-amino acids. For example, a stereoisomer of a naturally-occurring amino acid refers to the mirror image isomer of the naturally-occurring amino acid, i.e., the D-amino acid. Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

The term “amino acid modification” or “amino acid alteration” refers to a substitution, a deletion, or an insertion of one or more amino acids.

The term “backbone modification”, as used herein, refers to direct modification of amino acids constituting a peptide backbone with amino acid analogs, in which the backbone (main chain) refers to a main chain-or ring-shaped framework of amino acids constituting a peptide. The amino acid analog refers to an amino acid modified by substitution of hydrogen atoms on the nitrogen or a-carbon of the amino acid backbone.

The term “side-chain modification”, as used herein, refers to the modification of side-chains of amino acids by using a chemical material, in which the side-chains of amino acids refer to atomic groups branched from a main chain- or ring-shaped framework of amino acids constituting a peptide. Examples of the peptide side-chain modification may include amino group modification such as reductive alkylation; amidation with methyl acetimidate; alkylation with acetic anhydride; carbamylation of amino groups with cyanate; trinitrobenzylation of amino acids with 2,4,6-trinitrobenzene sulfonic acid (TNBS); alkylation of amino groups with succinic anhydride; and pyridoxylation with pyridoxal-5-phosphate followed by reduction with NaBH4.

The term “antibody” is inclusive of all species, including human and humanized antibodies and the antigenic target, can be from any species. Thus, an antibody, for example, which binds to an antigen “X” can be mouse anti-human X, human anti-human X; humanized anti-human X, goat anti-human X; goat anti-mouse X; rat anti-human X; mouse anti-rat X and the like. The combinations of antibody generated in a certain species against an antigen target, e.g. “X”, from another species, or in some instances the s0020ame species (for example, in autoimmune or inflammatory response) are limitless and all species are embodied in this invention. The term antibody is used in the broadest sense and includes fully assembled antibodies, monoclonal antibodies (including human, humanized or chimeric antibodies), polyclonal antibodies, multispecific antibodies (e.g., bispecific antibodies), and antibody fragments that can bind antigen (e.g., Fab′, F′ (ab)₂, Fv, single chain antibodies, diabodies), comprising complementarity determining regions (CDRs) of the foregoing as long as they exhibit the desired biological activity.

As used herein, an antibody that “specifically binds” to a target is intended to refer to a targeting ligand, e.g. an antibody that binds to a target with a K_{D} of 1×10⁻⁷ M or less, 5×10⁻⁸ M or less, 3×10⁻⁸ M or less, 1×10⁻⁸ M or less, 5×10⁻⁹ M or less.

The term “high affinity” for an antibody refers to an antibody having a K_{D} of 1×10⁻⁷ M or less, 5×10⁻⁸ M or less, 1×10⁻⁸ M or less, 5×10⁻⁹ M or less and 1×10⁻⁹ M or less for a target antigen. However, “high affinity” binding can vary for other antibody isotypes. For example, “high affinity” binding for an IgM isotype refers to an antibody having a K_{D} of 10⁻⁶ M or less, 10⁻⁷ M or less, 10⁻⁸ M or less.

The term “assay” used herein, whether in the singular or plural shall not be misconstrued or limited as being directed to only one assay with specific steps but shall also include, without limitation any further steps, materials, various iterations, alternatives etc., that can also be used. Thus, if the term “assay” is used in the singular, it is merely for illustrative purposes.

As used herein, the term “high-risk” refers to an enhanced risk of having one or more health problems such as birth defects, sequelae, ocular or mental disorder, ocular toxoplasmosis, congenital toxoplasmosis, and cerebral toxoplasmosis, for example. The term “risk” refers to the propensity of a subject to have one or more health problems.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to a delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

The terms “measuring” and “determining” are used interchangeably throughout, and refer to methods which include obtaining or providing a patient sample and/or detecting the level of a biomarker(s) in a sample. In one embodiment, the terms refer to obtaining or providing a patient sample and detecting the level of one or more biomarkers in the sample. In another embodiment, the terms “measuring” and “determining” mean detecting the level of one or more biomarkers in a patient sample. The term “measuring” is also used interchangeably throughout with the term “detecting.” In certain embodiments, the term is also used interchangeably with the term “quantitating.”

“Optional” or “optionally” means that the subsequently described event or circumstance can or cannot occur, and that the description includes instances where the event or circumstance occurs and instances where it does not.

A “reporter molecule”, a “label” or a “detectable label” are used interchangeably herein and refer to a composition detectable by spectroscopic, photochemical, biochemical, immunochemical, or chemical means. For example, useful labels include radio labeled molecules fluorophores, radiochemical, luminescent compounds, electron-dense reagents, enzymes (e.g., as commonly used in an ELISA), biotin, radioactive compounds, non-radioactive compounds, digoxigenin, or haptens and proteins which can be made detectable, e.g., by incorporating a label into the peptide.

The terms “sample,” “patient sample,” “biological sample,” and the like, encompass a variety of sample types obtained from a patient, individual, or subject and can be used in a diagnostic, prognostic and/or monitoring assay. The patient sample may be obtained from a healthy subject or a diseased patient. In certain embodiments, a sample that is “provided” can be obtained by the person (or machine) conducting the assay, or it can have been obtained by another, and transferred to the person (or machine) carrying out the assay.

Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, peripheral blood, serum, plasma, cord blood, amniotic fluid, cerebrospinal fluid, urine, saliva, stool and synovial fluid), solid tissue samples such as a biopsy specimen or tissue cultures or cells derived therefrom and the progeny thereof. In certain embodiment, a sample comprises cerebrospinal fluid. In a specific embodiment, a sample comprises a blood sample. In another embodiment, a sample comprises a plasma sample. In yet another embodiment, a serum sample is used.

The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

The terms “subject,” “individual,” and “patient” are used interchangeably herein to refer to a vertebrate, preferably a mammal, more preferably a human. Mammals include, but are not limited to mice, rats, simians, humans, farm animals, sport animals, and pets. Tissues, cells and their progeny of a biological entity obtained in vivo or cultured in vitro are also encompassed.

Any compositions or methods provided herein can be combined with one or more of any of the other compositions and methods provided herein.

Genbank and NCBI submissions indicated by accession number cited herein are incorporated herein by reference. All other published references, documents, manuscripts and scientific literature cited herein are incorporated herein by reference.

### BRIEF DESCRIPTION OF THE DRAWINGS

FIG. 1 is a flowchart to show case and control identification. Abbreviations: HIV, human immunodeficiency virus; TE, toxoplasmic encephalitis.

FIGS. 2A-2C are a series of plots showing the distribution of antibodies to MAG1_4.2 (FIG. 2A), MAG1_5.2 (FIG. 2B), and Toxoplasma (FIG. 2C) by cases and controls 2 years before the diagnosis of toxoplasmic encephalitis. The length of the box corresponds to the interquartile range with the upper boundary of the box representing the 75th percentile and the lower boundary the 25th percentile. The horizontal line in the box represents the median value. The whiskers represent the minimum and maximum. The scatter plot shows individual values. Log₂-transformed values for MAG1 data were used. Abbreviations: MAG1, matrix antigen 1.

FIG. 3 shows the efficacy of αPD-L1 mAb treatment against latent Toxoplasma infection. Nine-week-old BALB/c mice were infected i.p. with 400 tachyzoites of Pru strain. At 6 wpi, anti-PD-L1 antibodies or isotype controls were administrated to infected mice every 3 days for 2 weeks. Six weeks after the final injection, mice were sacrificed to count the number of tissue cysts in the brain. Compared to isotype control, the number of tissue cysts was reduced by 55% in mice treated with αPD-L1. P value is calculated by Student's t-test. Shown are the individual data points with a mean line.

FIG. 4 (includes FIGS. 4A-4E) shows leukocytes diminished in the meninges 6 weeks after αPD-L1 treatment. Representative images of CD3e (red), CD14 (green) labelling in whole-mount meninges (scale bar, 2,000 mm) for uninfected control (FIG. 4A), infected control (FIG. 4B), isotype (FIG. 4C), αPD-L1 (FIG. 4D and E). DAPI was used to identify nuclei (blue).

FIGS. 5A-5D show PD-L1 blockade on the suppression of glial and complement activation. Protein expression levels of GFAP (FIG. 5A), IBA1 (FIG. 5B), C1q (FIG. SC), and C4 (FIG. 5D) were compared in brain homogenates of mice that received αPD-L1 (n=6) and isotype control (n=5), alongside β-actin loading controls by western blot. The analysis was run on a 4-20% polyacrylamide gel under either reducing (FIGS. 5A, 5B, and 5C) or non-reducing conditions (FIG. 5D). Histograms indicate densitometric analysis of blots, expressed as means±SEM, with individual data points. The relative protein expression was assessed with MAG1 antibody levels measured after the treatment by Pearson's correlation analysis (n=11). Analysis was performed in three independent experiments. The dots show individual values from one representative experiment. Significance was determined with a student's t-test.

FIGS. 6A to 6C show changes in MAG1 antibody levels before and after the αPD-L1 or isotype treatment. Comparison of the MAG1 antibodies pre-and post-treatment for αPD-L1 (FIG. 6A) (n=7, p=0.066) and isotype (FIG. 6B) (n=5, p=0.153) mice. The number of tissue cysts enumerated in brains of αPD-L1-treated and isotype control mice at the end of therapy correlated with MAG1 antibody levels measured post-treatment (FIG. 6C). P values are calculated by paired t-test (FIGS. 6A and 6B) or Pearson's correlation analysis (FIG. 6C). Black dot. αPD-L1 mice; circle: isotype mice.

### DETAILED DESCRIPTION

The compositions described herein, have utility in predicting risk of subsequent diseases following Toxoplasma infection in multiple at-risk populations including patients with ocular toxoplasmosis or congenital toxoplasmosis, transplant recipients and other immunosuppressed patients with prior exposure to Toxoplasma. Exposure to Toxoplasma is common in the general population, but severe consequences only occur in some individuals. Prior to the disclosure herein, it has been difficult to identify the risk factors and predict the incidence of disease. For example, Toxoplasmic encephalitis (TE) is a brain infection with high morbidity and mortality. However, the clinical signs and symptoms of TE are nonspecific. The most important risk factor for TE is immunosuppression, although some Toxoplasma-infected AIDS patients never developed TE despite being severely immunocompromised. The poor predictive value of known risk factors for TE among Toxoplasma infected individuals and the challenges of making a definitive diagnosis argue for the need to develop robust pre-diagnostic markers for TE in order to better manage patients at risk. Management may include prophylactic administration of anti-Toxoplasma drugs, adjustments to immunosuppressive therapy, or enhanced clinical surveillance of patients for signs and symptoms of severe toxoplasmosis.

To address these problems, novel peptide antigens of the Toxoplasma cyst protein MAG1, were designed. Both peptides are chimeric constructs that do not exist in nature. Specifically, the peptides comprise discontinuous amino acid sequence of the MAG1 open reading frame. The amino acid sequence of MAG1_4.2 includes aa 342-361 and aa 422-452, with an added proline (P) between the discontinuous segments (EKQLRRVEPEHEDNTRVEAR/P/RALLEAKTKELVEPTSKEAEEARQILAEQAA; SEQ ID NO: 1) and MAG1_5.2 includes aa 66-82 and aa 107-148 (YENSEDVAVPSDSASTP/DCEEQQEQGDTTLSDHDFHSGGTEQEGLPETEVAHQHE TEEQ; SEQ ID NO: 2). The amino acid numbers are from accession number XP_002365700 and the slash (/) indicates the separation point between the two discontinuous amino acid regions.

An enzyme-linked immunoassay (ELISA assay) was designed using the peptides as solid phase antigens and measured serum antibodies that bind to the antigens. The results demonstrated the value of seroreactivity in the MAG1 peptide ELISA assays to predict the risk of toxoplasmic encephalitis (TE) in people living with HIV. It was hypothesized that the MAG1 peptides may have broader predictive value for other Toxoplasma-associated diseases. The at-risk populations includes patients with ocular toxoplasmosis and congenital toxoplasmosis, and transplant recipients and other immunosuppressed patients.

### Compositions

In certain embodiments, a composition comprises a peptide having an amino acid sequence of at least a 50%, 60%, 70%, 80%, 90% or 98% or 99% sequence identity to EKQLRRVEPEHEDNTRVEAR/P/RALLEAKTKELVEPTSKEAEEARQILAEQAA (SEQ ID NO: 1). In certain embodiments, the peptide comprises an amino acid sequence having at least a 75% sequence identity to EKQLRRVEPEHEDNTRVEAR/P/RALLEAKTKELVEPTSKEAEEARQILAEQAA (SEQ ID NO: 1). In certain embodiments, the peptide comprises an amino acid sequence having at least a 95% sequence identity to EKQLRRVEPEHEDNTRVEAR/P/RALLEAKTKELVEPTSKEAEEARQILAEQAA (SEQ ID NO: 1). In certain embodiments, the peptide comprises an amino acid sequence of

(SEQ ID NO: 1)
EKQLRRVEPEHEDNTRVEAR/P/RALLEAKTKELVEPTSKEAEEAR
QILAEQAA.

In certain embodiments, a composition comprises a peptide having an amino acid sequence of at least a 50% 60%, 70%, 80%, 90% or 98% or 99% sequence identity to YENSEDVAVPSDSASTP/DCEEQQEQGDTTLSDHDFHSGGTEQEGLPETEVAHQHET EEQ (SEQ ID NO: 2). In certain embodiments, the peptide comprises an amino acid sequence having at least a 75% sequence identity to YENSEDVAVPSDSASTP/DCEEQQEQGDTTLSDHDFHSGGTEQEGLPETEVAHQHET EEQ (SEQ ID NO: 2). In certain embodiments, the peptide comprises an amino acid sequence having at least a 95% sequence identity to YENSEDVAVPSDSASTP/DCEEQQEQGDTTLSDHDFHSGGTEQEGLPETEVAHQHET EEQ (SEQ ID NO: 2). In certain embodiments, the peptide comprises an amino acid sequence of

(SEQ ID NO: 2)
YENSEDVAVPSDSASTP/DCEEQQEQGDTTLSDHDFHSGGTEQEGL
PETEVAHQHETEEQ.

In certain embodiments, a composition comprises peptides having a 50% 60%, 70%, 80%, 90% or 98% or 99% sequence identity to SEQ ID NO: 1 and 2. In certain embodiments, a composition comprises peptides having a 75% sequence identity to SEQ ID NO: 1 and 2. In certain embodiments, a composition comprises peptides having a 95% sequence identity to SEQ ID NO: 1 and 2. In certain embodiments, a composition comprises peptides comprising SEQ ID NO: 1 and 2.

In certain embodiments, peptides comprising SEQ ID NOS: 1 or 2 comprise one or more modifications. These include, for example, naturally-occurring amino acids, non-naturally occurring amino acids, deletions, substitutions, insertions and combinations thereof.

Naturally-occurring amino acids are those encoded by the genetic code, as well as those amino acids that are later modified, e.g., hydroxyproline, γ-carboxyglutamate and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” are unnatural amino acids that have the same basic chemical structure as naturally-occurring amino acids, i.e., an a carbon that is bound to a hydrogen, a carboxyl group, an amino group, but have modified R (i.e., side-chain) groups or modified peptide backbones, e.g., homoserine, norleucine, methionine sulfoxide, methionine methyl sulphonium. “Amino acid mimetics” refer to chemical compounds that have a structure that is different from the general chemical structure of an amino acid, but that functions in a manner similar to a naturally-occurring amino acid.

With respect to amino acid sequences, one of skill in the art will recognize that individual substitutions, additions, or deletions to a peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. The chemically similar amino acid includes, without limitation, a naturally-occurring amino acid such as an L-amino acid, a stereoisomer of a naturally occurring amino acid such as a D-amino acid, and an unnatural amino acid such as an amino acid analog, amino acid mimetic, synthetic amino acid, N-substituted glycine, and N-methyl amino acid.

Conservative substitution tables providing functionally similar amino acids are well known in the art. For example, substitutions may be made wherein an aliphatic amino acid (e.g., G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, e.g., E or D, may be substituted with its uncharged counterpart, e.g., Q or N, respectively; or vice versa. Each of the following eight groups contains other exemplary amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

In certain embodiments, amino acid modifications can be made based on the relative similarity of amino acid side-chain substituents, such as hydrophobicity, hydrophilicity, charge, size, and the like.

Accordingly, in certain embodiments, the peptides of the present disclosure, e.g. SEQ ID NOS: 1 or 2, include peptide modifications having a sequence that include one or more amino acid residues different from those of the amino acid sequence of the peptides embodied herein.

In certain embodiments, arbitrary amino acids are added at the N-terminus or C-terminus of the peptides. For example, a peptide prepared by adding 1 to 5, or 1 to 10, or 1 to 15 amino acids at the N-terminus or C-terminus of the peptide.

The peptides may be chemically modified or protected with an organic group at the N-terminus and/or C-terminus, or may be modified by adding amino acids at the peptide terminus. In particular, since chemically synthesized peptides have charged N-terminus and C-terminus, N-terminal acetylation, N-terminal methylation, or/and C-terminal amidation may be performed, or D-amino acid introduction, peptide bond modification such as CH₂—NH, CH₂—S, CH₂—S=0, CH₂—CH₂, backbone modification, or side-chain modification may be included in order to remove the charge, but is not limited thereto. Methods of preparing peptidomimetic compounds are well known in the art, for example, referring to a description in Quantitative Drug Design, C.A. Ramsden Gd., Choplin Pergamon Press (1992).

The present peptides may be derivatized by the attachment of one or more chemical moieties to the peptide sequence. Chemical modification of one or more residues may be achieved by chemically derivatizing a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides which contain one or more naturally occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine.

Desirable chemical moieties can be attached to the peptides at amino acid residues having a free amino group such as lysine residues and the N-terminal amino acid residue. Those having a free carboxyl group may include aspartic acid residues, glutamic acid residues, and the C-terminal amino acid residue.

Replacement of naturally occurring amino acids with a variety of uncoded or modified amino acids such as D-amino acids and N-methyl amino acids may also be used to modify peptides.

It may also be desirable to use derivatives of the peptides that are conformationally constrained. Conformational constraint refers to the stability and preferred conformation of the three-dimensional shape assumed by a peptide. Conformational constraints include local constraints, involving restricting the conformational mobility of a single residue in a peptide; regional constraints, involving restricting the conformational mobility of a group of residues, which residues may form some secondary structural unit; and global constraints, involving the entire peptide structure.

The active conformation of the peptide may be stabilized by a covalent modification, such as cyclization or by incorporation of gamma-lactam or other types of bridges. For example, side chains can be cyclized to the backbone so as to create a L-gamma-lactam moiety on each side of the interaction site. Cyclization also can be achieved, for example, by formation of cysteine bridges, coupling of amino and carboxy terminal groups of respective terminal amino acids, or coupling of the amino group of a Lys residue or a related homolog with a carboxy group of Asp, Glu or a related homolog. Coupling of the alpha-amino group of a polypeptide with the epsilon-amino group of a lysine residue, using iodoacetic anhydride, can be also undertaken.

Another approach is to include a metal-ion complexing backbone in the peptide structure. Typically, the preferred metal-peptide backbone is based on the requisite number of particular coordinating groups required by the coordination sphere of a given complexing metal ion. In general, most of the metal ions that may prove useful have a coordination number of four to six. The nature of the coordinating groups in the peptide chain includes nitrogen atoms with amine, amide, imidazole, or guanidino functionalities; sulfur atoms of thiols or disulfides; and oxygen atoms of hydroxy, phenolic, carbonyl, or carboxyl functionalities. In addition, the peptide chain or individual amino acids can be chemically altered to include a coordinating group, such as for example oxime, hydrazino, sulfhydryl, phosphate, cyano, pyridino, piperidino, or morpholino. The peptide construct can be either linear or cyclic, however a linear construct is typically preferred. One example of a small linear peptide is Gly-Gly-Gly-Gly that has four nitrogens (an N4 complexation system) in the backbone that can complex to a metal ion with a coordination number of four.

Another approach is to use bifunctional crosslinkers, such as N-succinimidyl 3-(2 pyridyldithio) propionate, succinimidyl 6-[3-(2 pyridyldithio) propionamido]hexanoate, and sulfosuccinimidyl 6-[3-(2 pyridyldithio) propionamido]hexanoate.

### Utility

The peptides embodied herein have many useful utilities. For example, diagnosing Toxoplasma-associated diseases, immune detection assays (e.g. ELISA), in vivo imaging and the like.

Accordingly, in certain embodiments a method of diagnosing Toxoplasma-associated disease in a subject comprises obtaining a biological sample from the subject; culturing the biological sample with a modified Toxoplasma cyst peptide; measuring the amount of antibodies binding to the modified Toxoplasma cyst peptide. In certain embodiments, the modified Toxoplasma cyst peptide comprises a Toxoplasma matrix antigen 1 (MAG1) peptide comprising one or more amino acid deletions, amino acid substitutions, amino acid insertions or combinations thereof.

In certain embodiments, the modified Toxoplasma cyst peptides are utilized in an immunoassay. Techniques for the assays contemplated herein are known in the art and include, for example, radioimmunoassay (RIA), enzyme-linked immunosorbent assay (ELISA), sandwich assays, ELISpot, blots and the like. Immunoassays often utilize a labeling agent to specifically bind to and label the binding complex formed by the antibody and the target protein (antigen). The labeling agent may itself be one of the moieties comprising the antibody/target protein complex, or may be a third moiety, such as another antibody, that specifically binds to the antibody/target protein complex. A label may be detectable by spectroscopic, photochemical, biochemical, immunochemical, electrical, optical or chemical means. Examples include, but are not limited to, magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase, and others commonly used in an ELISA), and colorimetric labels such as colloidal gold or colored glass or plastic (e.g., polystyrene, polypropylene, latex, etc.) beads.

In some cases, the labeling agent is a second antibody (e.g., an IgG antibody) bearing a detectable label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species.

Immunoassays for detecting a specific Toxoplasma antibody of interest, e.g., an IgG specifically immune-reactive against the peptides embodied herein, from samples may be either competitive or noncompetitive. A typical specific IgG immunoassay is a noncompetitive immunoassay in which the amount of captured target IgG is directly measured. In one “sandwich” assay, for example, one or more of the Toxoplasma peptides, inclusive of the modified peptides embodied herein, can be bound or immobilized directly to a solid substrate (such as the surface of a plate). The immobilized peptides can then capture the specific antibodies in the biological samples. The antibody/target protein complex thus immobilized is then bound by a labeling agent, such as a second or third antibody bearing a label, as described above.

The solid surface is typically glass or a polymer, the most commonly used polymers being cellulose, polyacrylamide, nylon, polystyrene, polyvinyl chloride or polypropylene. The solid supports may be in the form of tubes, beads, spheres, discs of microplates, or any other surface suitable for conducting an immunoassay: The binding processes are well known in the art and generally consist of cross-linking covalently binding or physically adsorbing the peptide to the substrate. An aliquot of the sample to be tested is then added to the solid phase complex and incubated for a period of time sufficient (e.g. 2-120 minutes or where more convenient, overnight) and under suitable conditions (e.g. for about 20° C. to about 40° C.) to allow binding of the antibody. Following the incubation period, the antibody-peptide solid phase is washed and dried and incubated with a second antibody specific for a portion of the bound antibody. The second antibody is linked to a reporter molecule or detectable molecule which is used to indicate the binding of the second antibody to the complex.

Other assay formats include liposome immunoassays (LIA), which use liposomes designed to bind specific molecules (e.g., antibodies) and release encapsulated reagents or markers. The released chemicals are then detected according to standard techniques (see, Monroe et al., Amer. Clin. Prod. Rev., 5:34-41 (1986)).

In certain embodiments, the modified Toxoplasma cyst peptides are immobilized to a solid medium. The biological sample is then incubated with the peptides. Antibodies which specifically bind to the modified Toxoplasma cyst peptides are detected by a reporter molecule or detectable label, e.g. attached to a secondary antibody.

In certain embodiments, the modified Toxoplasma cyst peptides are in a semi-solid medium e.g. a gel. In certain embodiments, the modified Toxoplasma cyst peptides are in solution. For example, the subjects antibodies may be the ones attached, to for example a bead or a solid substrate and the peptides are in solution allowing for capture by Toxoplasma cyst antibodies.

By “reporter molecule” or “detectable label” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules. In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-antigen complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.

Alternately, fluorescent compounds, such as fluorescein and rhodamine, may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. The fluorescent labeled antibody is allowed to bind to the first antibody-antigen complex. After washing off the unbound reagent, the remaining tertiary complex is then exposed to the light of the appropriate wavelength the fluorescence observed indicates the presence of the antigen of interest. Immunofluorescene and enzyme immunoassay techniques are both very well established in the art and are particularly preferred for the present method. However, other reporter molecules, such as radioisotope, chemiluminescent or bioluminescent molecules, may also be employed.

There are a range of other detection systems which may be employed including colloidal gold and all such detection systems are encompassed by the present disclosure.

### Kits

Any of the compositions described herein may be comprised in a kit. In a non-limiting example, modified Toxoplasma cyst peptides may be comprised in a kit. The kits may also comprise a suitably aliquoted of a MAG1 antibody and/or a T. gondii antibody and, in some cases, one or more additional agents. The component(s) of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing modified Toxoplasma cyst peptides and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. A modified Toxoplasma cyst peptide may be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means.

In certain embodiments, the kit further comprises one or more secondary antibodies or antigen binding fragments thereof, for detecting antibodies bound to the modified Toxoplasma cyst peptides. In certain embodiments, the one or more secondary antibodies or antigen binding fragments thereof, comprise a detectable label. In certain embodiments, the reporter molecule or detectable label comprises fluorophores, radiochemical, luminescent compounds, electron-dense reagents, enzymes, biotin, radioactive compounds, non-radioactive compounds, digoxigenin, or haptens. In certain embodiments, the kit optionally comprises a capture antibody which specifically binds to the one or more modified Toxoplasma cyst peptides. In certain embodiments, the capture antibody binds to an epitope distinct to that of an antibody present in a biological sample of a subject infected with a Toxoplasma parasite. In certain embodiments, Toxoplasma parasite is Toxoplasma gondii.

In certain embodiments, the kits may include a MAG1 antibody and/or a T. gondii antibody.

See also Xiao et al., Serological Responses to Toxoplasma gondii and Matrix Antigen 1 Predict the Risk of Subsequent Toxoplasmic Encephalitis in People Living With Human Immunodeficiency Virus (HIV), Clinical Infectious Diseases 2021; Clin Infect Dis. 2021 Oct. 5;73(7):e2270-e2277.

### EXAMPLES

The following non-limiting Examples serve to illustrate selected embodiments of the disclosure. It will be appreciated that variations in proportions and alternatives in elements of the components shown will be apparent to those skilled in the art and are within the scope of embodiments of the present disclosure.

### Example 1: Serological Responses to Toxoplasma gondii and Matrix Antigen 1 Predict the Risk of Subsequent Toxoplasmic Encephalitis in People Living With Human Immunodeficiency Virus (HIV)

Previously, the inventors developed a serological assay for antibodies to the Toxoplasma matrix antigen 1 (MAG1) [8]. The level of MAG1 antibodies was highly correlated with the number of tissue cysts in the brain of infected mice and with signs of chronic infection, including lower body weight, behavioral changes, altered gene expression, and immune activation [9]. Herein samples that had been collected 2 years prior to the diagnosis of TE from Toxoplasma seropositive people living with human immunodeficiency virus (HIV: PLWH) were identified to evaluate the predictive value of serum antibodies to MAG1 and the whole Toxoplasma organism for development of TE, alone or in combination. The choice of two years was intended to balance a reasonably long lead time before diagnosis and a clinically pertinent interval where therapeutic interventions, such as administration of anti-microbial prophylaxis, could be deployed.

### Methods

### Study Population

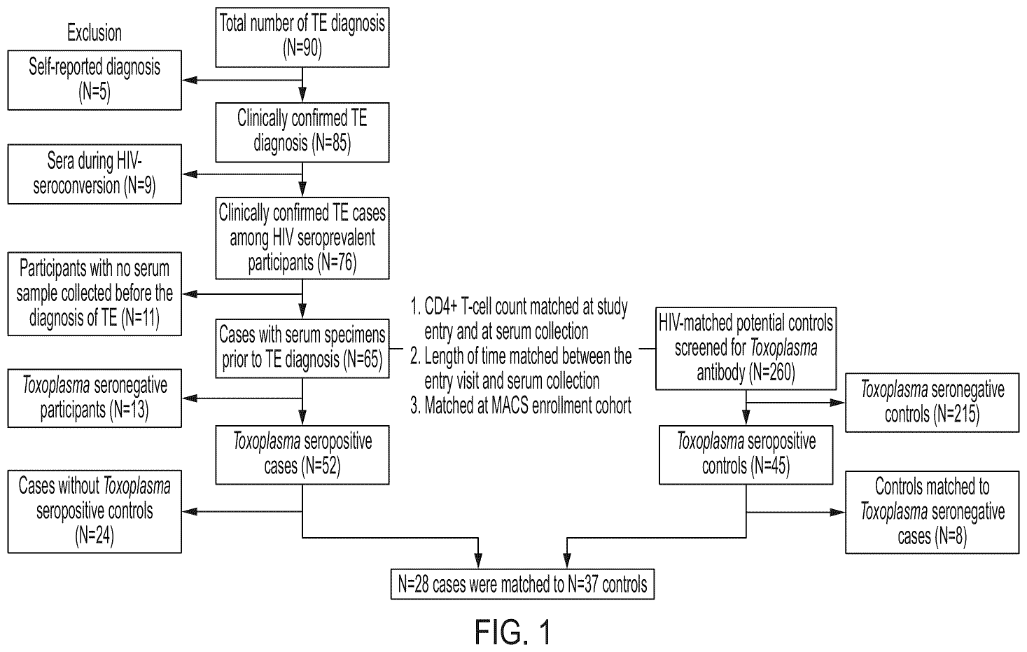

The study was a matched case-control design nested within the Multicenter AIDS Cohort Study (MACS), a prospective study of HIV/AIDS in men who have sex with men (MSM) in the United States, begun in 1984 [10, 11]. MACS recruited men from 4 US cities (Baltimore/Washington D.C.; Chicago, Illinois; Pittsburgh, Pennsylvania; Los Angeles, California) and had 4 enrollment cohorts: 1984-1985, 1987-1991, 2001-2003, and 2010-2018. MACS is a longitudinal study, where data and serum samples were collected at study entry and semiannual visits via interviewer-administered and computer-assisted questionnaires and physical examinations. Clinical outcomes were reported at the time of diagnosis by the attending physician. The present study is restricted to PLWH. FIG. 1 is a flowchart showing case and control identification. There were 90 cases of TE reported in the MACS outcomes database between 1984 and 2002, diagnosed by clinical signs and symptoms, radiologic criteria for TE, or a self-report of TE. Cases were excluded if the diagnosis was based on self-report (n=5), sera from recent HIV seroconverters (n=9), or prediagnostic serum samples were not available (n=11). After exclusions, 65 cases were screened for Toxoplasma antibodies. Toxoplasma seropositivity was an eligibility criterion because TE is the result of reactivation of a prior Toxoplasma infection. Thirteen of the 65 cases were seronegative and were excluded from the study, leaving a total of 52 cases.

Because the risk of Toxoplasma reactivation is related to the degree and duration of immunosuppression, controls were matched to cases for (1) CD4+T-cell count (±100 cells/mm³ cells) at MACS enrollment and at the time of serum collection, and (2) length of time (±3.5 years) between MACS enrollment and serum collection. To account for potential demographic and/or socioeconomic differences, controls were also matched for the MACS enrollment cohort. For each case, 4 controls matched on the criteria above were selected and then screened for Toxoplasma antibodies. Among 260 eligible controls (for the 65 cases), 45 (17.3%) were seropositive for Toxoplasma antibody, consistent with the expected seroprevalence of Toxoplasma in this population [12]. Among the 45 eligible Toxoplasma seropositive controls, 37 were matched to 28 TE cases. Some cases had more than 1 control serum samples (20 cases had 1 control, 7 cases had 2 controls, and 1 case had 3 controls). The 28 TE cases included in the final analysis were drawn from the first 2 MACS cohorts. Twenty-four of the 52 Toxoplasma-seropositive TE cases were not included in the final analysis because their HIV disease matched controls were Toxoplasma-seronegative. All controls were diagnosed with an AIDS-defining condition, other than TE, subsequent to the time of serum collection.

### Serological Assays

Sera were analyzed in a blinded manner for antibodies to whole Toxoplasma organism, MAG1, and strain-specific antigens derived from 3 strains. Total anti-Toxoplasma IgG antibodies were measured using a commercial ELISA kit (IB19213, IBL America, Minneapolis, Minnesota, USA) according to the manufacturer's protocol. Antibody against Toxoplasma cyst antigen MAG1 was measured using a peptide ELISA as previously described with newly designed peptide antigens [8]. The new peptides consisted of discontinuous amino acid sequence of the MAG1 open reading frame. The amino acid sequence of MAG1_4.2 includes aa 342-361 and aa 422-452, with an added proline (P) between the discontinuous segments (EKQLRRVEPEHEDNTRVEAR/P/RALLE AKTKELVEPTSKEAEEARQILAEQAA; SEQ ID NO: 1) and MAG1_5.2 includes aa 66-82 and aa 107-148 (YENSEDVAVPSDSASTP/DCEEQQEQGDTTLSDHDFHSGGTEQEGLPETEVAHQHETEEQ; SEQ ID NO: 2). The amino acid numbers are from accession number XP_002365700 and the slash (/) indicates the separation point between the 2 discontinuous amino acid regions. Peptides were chemically synthesized to 90% purity (GenScript, Piscataway, New Jersey, USA).

The Toxoplasma strain type of patients infected with was determined by a previously developed serological assay [13]. Six polymorphic peptides (GRA5-II, GRA6-I, GRA6-II, GRA6-III, GRA7-II, and GRA7-III) specific to 3 clonal parasite lineages and derived from 3 dense granule antigens (GRA5, GRA6, and GRA7) were used for serological typing, as described previously [13]. Briefly, the Toxoplasma serotype was determined by a 2-step screening process: first, serum samples were tested to distinguish type II from type I/III infection using 5 peptides (GRA6-I, GRA6-III, GRA5-II, GRA6-II and GRA7-II). Second, type III was distinguished from type I infection using the GRA7-III peptide.

### Statistical Analysis

For each participants, MAG1_4.2 and MAG1_5.2 antibodies were measured 3 times on 3 different days, and results were expressed as the average optical density (OD) value of the replicates. Because antibody results were not normally distributed within the data set, median values with interquartile ranges (IQR) were reported, and comparisons between cases and controls were based on Wilcoxon signed-rank statistics for continuous covariates. Differences in proportions across persons with and without TE were assessed by Mantel-Haenszel statistics. In order to evaluate the seroprevalence of MAG1 antibodies, receiver operating characteristic (ROC) curve analysis was performed to determine an optimal cutoff point. The area under the curve (AUC) was .78 (.67, .89) for MAG1_4.2 and .54 (.39, .68) for MAG1_5.2. According to Youden's J Index, ROC-derived cutoff was an OD value of .046 for MAG1_4.2, and .063 for MAG1_5.2. The robustness of the cutoffs was further assessed by sensitivity analyses performed using cutoffs 10% above and below the ROC-defined optimum. The odds ratios (ORs) were calculated for having a positive MAG1 test (reactivity greater than the cutoffs) using either the Mantel-Haenszel test or conditional logistic regression, with or without adjustment for age and race. The Mantel-Haenszel test was the primary analysis because the small sample size made estimates from conditional logistic regression less reliable. Conditional logistic regression analysis was also used to assess the association between antibody levels and TE. Sensitivity, specificity, positive predictive values (PPV), negative predictive values (NPV), positive likelihood ratio (LR⁺), and negative likelihood ratio (LR⁻) were estimated for each marker or combination of markers. For analyses involving the combined use of markers, individuals were considered seropositive if they were seropositive for MAG1_4.2 or MAG1_5.2, or the level of Toxoplasma IgG was in the upper tertile of seroreactivity. P values <.05 were considered statistically significant. All statistical calculations were performed using SAS software (SAS, Cary, North Carolina, USA), and graphs were made in GraphPad Prism 8 (GraphPad, San Diego, California, USA).

### Results

### Participant Population

Demographic characteristics of matched cases and controls were similar at the time of serum collection (Table 1). Of note, the dates of the study preceded the introduction of antiretroviral therapy (ART), with only 1 case and 3 controls having a history of ART use. The use of anti-Toxoplasma drug prophylaxis was rare with only 1 case receiving pyrimethimine before the diagnosis of TE. Among the 28 cases and 37 matched controls, confounding factors that potentially could have contributed to disease status were matched accurately using the matching criteria described in Methods section (Table 1). The median time between serum collection and TE diagnosis was 2.16 years (IQR: 1.9-3 years) for cases. For controls, the median time was 2.93 years (IQR: 1.8-4 years) between serum collection and the time corresponding to the TE diagnosis of the matched case.

### Antibody Distributions in Cases and Controls Prior to TE Diagnosis

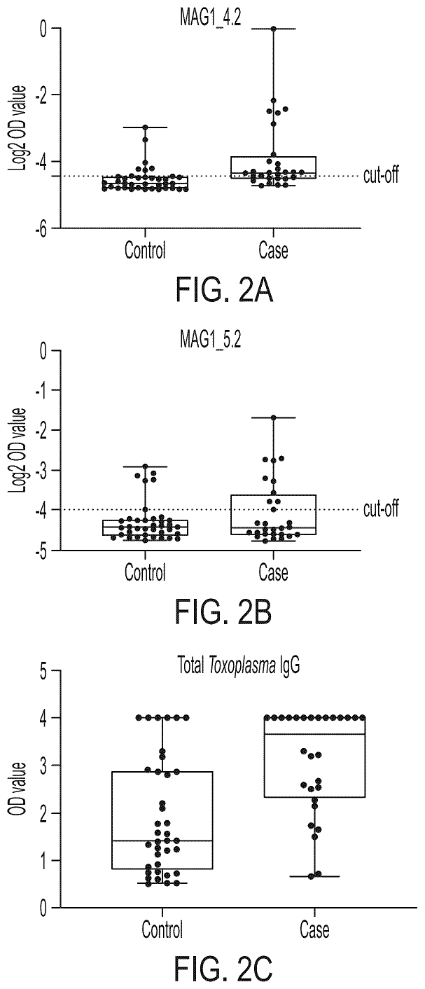

The median level of antibody to MAG1_4.2 was significantly higher in cases than controls (P<.001, FIG. 2A), but this was not true for antibody to MAG1_5.2 (FIG. 2B). By design all cases and controls were seropositive for Toxoplasma antibodies. However Toxoplasma seroreactivity was significantly higher in cases compared to controls (3.65 vs. 1.43 OD units) (P<0.001, FIG. 2C).

### MAG1 Seropositivity in Relation to the Diagnosis of TE

MAG1 seropositivity was significantly associated with the risk of TE (Table 2). MAG1_4.2 seropositivity was 67.8% and 16.2% and MAG1_5.2 seropositivity was 35.7% and 13.5% for cases and controls, respectively. Seropositivity in either test was 71.4% for cases and 27.0% for controls (Mantel-Haenszel OR, 6.71; 95% confidence interval [CI]: 1.67, 26.97).

Conditional logistic regression analysis also showed the association between MAG1 seropositivity and the risk of TE. In an unadjusted model, the odds of MAG1 4.2 seropositive was 19.3 (95% CI: 2.52, 145.41) times higher among men with TE diagnosed within a 2-year time frame compared to that among controls. Similarly, after adjusting for age and race, the odds of MAG1_5.2 seropositive was 6.71 (95% CI: 1.12, 40.28) times higher among men who subsequently developed TE compared to that among controls.

### Sensitivity Analyses of the ROC-derived Seroprevalence for the Diagnosis of TE

To ensure the robustness of these findings, sensitivity analyses-testing the effect of different “cutoffs” on the observed findings, was conducted. Cutoffs that were 10% above and below the ROC cut-off for MAG1_4.2 and MAG1_5.2 seropositivity, were chosen. The results showed that the cutoff for MAG1_5.2 was robust with minimal differences in odds ratios compared to that obtained using the ROC cutoff. Although the cutoff for MAG1_4.2 was more sensitive to variation, the direction of the association was the same for all cut-off values.

### Antibody Levels in Relation to the Diagnosis of TE

Levels of antibodies were also analyzed in relation to the odds of developing TE (Table 3). Using log2-transformed OD values, it was found that the odds of being a case increased 18.51 (95% CI: 1.41, 242.7) times per doubling of the MAG1_4.2 antibody OD value (P=.026). For MAG1_5.2, the odds of being a case increased 2.29 (95% CI: .78, 6.71) times per doubling of the OD value (P=.131). For each OD unit increase of Toxoplasma antibodies, the odds of developing TE increased 2.91 (95% CI: 1.48, 5.72) fold (P=.002).

### Predictive Value of Toxoplasma IgG and MAG1 Antibodies

To assess the clinical usefulness of the serological tests, sensitivity, specificity, positive predictive value (PPV), negative predictive value (NPV), positive likelihood ratio (LR⁺), and negative likelihood ratio (LR⁻) (Table 4), were calculated. For the MAG1 assays the ROC cut point for seropositivity was used, and for the Toxoplasma antibody assay, a positive test was defined as seroreactivity in the upper tertile of OD values. The single marker with the highest sensitivity was MAG1_4.2 (68%) and sensitivity increased when the marker was combined with the other 2 markers (89%). Specificity of single markers was 81˜86% and that of combined markers was 68-78%. Overall, PPVs were comparable for different single markers or combination of markers (67˜76%). Combined markers generally had better NPVs compared to single markers (74˜89% vs 64˜78%). The LR⁺ranged between 2.57 and 4.25, with MAG1_4.2 having the highest LR⁺. The LR⁻ranged between .16 and .74, with the combination of 3 markers having the lowest LR⁻. Based on sensitivity and LR⁻criteria, the combination of 3 markers had the best performance characteristics, with a sensitivity of 89% and an LR⁻of .16.

### Toxoplasma Serotypes in Relation to the Diagnosis of TE

Because the strain of parasite may influence pathological outcome [2], the infecting strain was determined serologically using a previously developed assay (Table 5) [13]. Among cases, 9 (32%) were type II, 1 (4%) was type III, 3 (11%) were atypical, and 15 (53%) were indeterminate. Among controls, 3 (8%) were type I, 8 (22%) were type II, 1 (3%) was atypical, and 25 (67%) were indeterminate. There were no statistically significant differences among serotypes by case and control status (χ² value 6.436, P=.17). Serotype II was the most common strain among cases and controls, with a prevalence of 32% and 22% in cases and controls, respectively.

### Discussion

Although profound immunosuppression is the strongest predictor for development of TE, the fact that only one-third of Toxoplasma seropositive AIDS patients during the pre-ART era developed TE indicates the importance of other risk factors [1]. The pre-diagnostic value of serological responses to Toxoplasma and matrix antigen 1 were explored in a matched case-control study of PLWH. It was found that antibodies to MAG1 and high levels of Toxoplasma antibodies are present more commonly in PLWH who developed TE compared with controls approximately 2 years prior to diagnosis. The combination of the serological assays had high sensitivity for predicting the risk of subsequent TE. In a high-risk population, tests with high sensitivity may have clinical value for screening even when specificity is only modest.

This is the first report of antibodies to MAG1 serving as a predictive biomarker for TE in PLWH. As described previously [9], measurement of MAG1 antibodies offers an approach for indirectly ascertaining parasite burden. Tissue cysts, the hallmark of chronic Toxoplasma infection, are predominantly located in the brain, making direct measurement of parasite burden difficult. Using highly sensitive quantitative polymerase chain reaction (qPCR) assays that target the multicopy B1 gene and AF146527 element, Toxoplasma DNA can be detected in cerebral spinal fluid (CSF) and blood from symptomatic patients [14, 15]. The utility of these assays is generally limited to the time of diagnosis. In contrast, antibodies to MAG1 are predictive of TE approximately 2 years before the clinical diagnosis in PLWH and can be measured in readily available body fluids such as serum.

Serologic measurement of Toxoplasma associated antibodies is a noninvasive method for predicting the risk of TE. Although the use of ART has greatly reduced the incidence of TE in PLWH, they still remain at risk [16-20]. A systematic review and meta-analysis reported that even after the introduction of ART, TE accounted for 6% of hospital admissions among AIDS-related illnesses for PLWH [20]. One case from the current study progressed to TE despite receiving combination ART. In many countries where combination ART is not readily available or there are issues of medication adherence, Toxoplasma remains an important opportunistic infection [21]. In view of the rapidly progressive and fatal outcome of Toxoplasma reactivation [22, 23], early diagnosis is also crucial for other at-risk populations. For instance, these tests could be useful in prevention of ocular toxoplasmosis in individuals with ocular involvement or toxoplasmosis in organ and stem cell transplant recipients [23-25]. Congenital toxoplasmosis usually results from primary infection during pregnancy. However, mothers infected before conception are also at risk of transmitting this disease to the fetus in the setting of immunodeficiency [26-29]. In clinical practice, the ability to serologically identify individuals whose infections might progress to severe disease should allow closer clinical monitoring and, where appropriate, treatment with aggressive prophylactic therapy.

To conclude, relatively high levels of Toxoplasma antibodies combined with the presence of MAG1 antibodies in PLWH can be highly predictive of TE 2 years prior to the clinical diagnosis. Studies employing more participants, as well as participants from other at-risk populations, are required to further test the predicative value of these antibodies.

TABLE 1
Characteristics Between Matched Cases and Controls

| | Control | | Case | | P |
|---|---|---|---|---|---|
| Variable | (n = 37) | | (n = 28) | | value |
| Detectable viral load | | | | | .32 |
| (HIV RNA >50 copies/mL), | | | | | |
| n (%) | | | | | |
| Yes | 14 | (38) | 12 | (43) | |
| No | 1 | (3) | 0 | (0) | |
| Not available | 22 | (59) | 16 | (57) | |
| Age (years), median (IQR) | 38 | (34-45) | 36 | (34-38) | .07 |
| Race/ethnicity, n (%) | | | | | .03 |
| White | 34 | (92) | 23 | (82) | |
| Black | 1 | (3) | 5 | (18) | |
| Other | 2 | (5) | 0 | (0) | |
| CD4+ T-cell count at study | 533 | (451-642) | 544 | (407-677) | .18 |
| entry (cells/mm3), median | | | | | |
| (IQR) | | | | | .24 |
| CD4+ T-cell count at serum | 313 | (176-477) | 303 | (155-479) | |
| collection (cells/mm3), | | | | | |
| median (IQR) | | | | | |
| Time between study entry | 2.6 | (1.4-5.1) | 3.2 | (1.3-5.1) | .16 |
| and serum collection (years), | | | | | |
| median (IQR) | | | | | |
| Anti-HIV therapy, n (%) | | | | | .18 |
| No therapy | 16 | (43) | 15 | (53) | |
| Mono therapy (simply ART) | 5 | (14) | 2 | (7) | |
| ART therapy | 3 | (8) | 1 | (4) | |
| (combination ART) | | | | | |
| Data not available | 13 | (35) | 10 | (36) | |
| Pyrimethamine prophylaxis, | | | | | .32 |
| n (%) | | | | | |
| No | 37 | (100) | 27 | (96) | |
| Yes | 37 | (100) | 1 | (4) | |

P values were obtained by nonparametric Wilcoxon signed-rank test or Mantel-Haenszel test, as appropriate. Abbreviations: ART, antiretroviral therapy; HIV, human immunodeficiency virus; IQR, interquartile range.

TABLE 2
Mantel-Haenszel Analysis of MAG1 Seropositivity in Relation
to Toxoplasmic Encephalitis

| | Cases | | Controls | | | |
|---|---|---|---|---|---|---|
| MAG1 | (n = 28) | | (n = 37) | | | P |
| Assay | n | % | n | % | OR (95% CI) | value |
| MAG1_4.2 | | | | | 25.0 | <.001 |
| | | | | | (3.14 -199.18) | |
| Positive | 19 | 67.8 | 6 | 16.2 | | |
| (>.046) | | | | | | |
| Negative | 9 | 32.2 | 31 | 83.8 | | .033 |
| (<.046) | | | | | | |
| MAG1_5.2 | | | | | 3.6 | |
| | | | | | (.95-13.42) | |
| Positive | 10 | 35.7 | 5 | 13.5 | | |
| (>.063) | | | | | | |
| Negative | 18 | 64.3 | 32 | 86.5 | | |
| (<.063) | | | | | | |
| Combined^{a} | | | | | 6.7 | .001 |
| | | | | | (1.67-26.97) | |
| Positive | 20 | 71.4 | 10 | 27.0 | | |
| Positive | 8 | 28.6 | 27 | 73.0 | | |

Abbreviations: CI, confidence interval; MAG1, matrix antigen 1; OR, odds ratio.
^{a}Seropositivity in either MAG1_4.2 or MAG1_5.2 test.

TABLE 3
Conditional Logistic Regression Analysis of Increase in Levels of
MAG1 and Toxoplasma Antibodies in Relation to Toxoplasmic Encephalitis

| | | | Multivariate | | | | | |
|---|---|---|---|---|---|---|---|---|
| | Univariate | | MAG1_4.2 | | MAG1_5.2 | | Toxoplasma IgG | |
| | OR (95% CI) | P | OR (95% CI) | P | OR (95% CI) | P | OR (95% CI) | P |
| MAG1_4.2^{a} | 7.56 | .030 | 18.51 | .026 | | | | |
| | (1.2, 47) | | (1.4, 242) | | | | | |
| MAG1_5.2^{a} | 2.07 | .129 | | | 2.29 | .131 | | |
| | (.8, 5.3) | | | | (.78, 6.71) | | | |
| Toxoplasma | 2.45 | .004 | | | | | 2.91 | .002 |
| IgG | (1.3, 4.5) | | | | | | (1.5, 5.7) | |
| Age | .95 | .237 | .91 | .113 | .95 | .188 | .9 | .089 |
| | (.9, 1.0) | | (.8, 1.0) | | (.87, 1.0) | | (.8, 1.0) | |
| White^{b} | .31 | .180 | .1 | .139 | .35 | .284 | .09 | .095 |
| | (.06, 1.7) | | (.01, 2.1) | | (.05, 2.4) | | (.01, 1.5) | |

Abbreviations: CI, confidence interval; IgG, immunoglobulin G; MAG1, matrix antigen 1; OR, odds ratio.
^{a}Per doubling.
^{b}Reference: Black/Other.

TABLE 4
Sensitivity, Specificity, PPV, NPV, LR+, and LR− of Serological
Markers for Prediction of Toxoplasmic Encephalitis

| Markers | Sensitivity | Specificity | PPV | NPV | LR+ | LR− |
|---|---|---|---|---|---|---|
| MAG1_ | 68 | 84 | 76 | 78 | 4.25 | 0.38 |
| 4.2 | (48~84)^{a} | (68~94) | (59~87) | (66~86) | (1.9~9.1) | (.2~.7) |
| MAG1_ | 36 | 86 | 67 | 64 | 2.57 | 0.74 |
| 5.2 | (19~56) | (71~95) | (44~84) | (57~71) | (1.0~6.9) | (.6~1.0) |
| Third | 57 | 81 | 70 | 71 | 3.00 | 0.53 |
| tertile | (37~76) | (65~92) | (52~83) | (61~80) | (1.4~6.3) | (.3~.8) |
| of Toxo | | | | | | |
| IgG^{b} | | | | | | |
| MAG1_ | 86 | 70 | 69 | 87 | 2.87 | 0.2 |
| 4.2 or | (67~96) | (53~84) | (56~78) | (72~94) | (1.7~4.8) | (.1~.5) |
| third | | | | | | |
| tertile | | | | | | |
| of | | | | | | |
| Toxo | | | | | | |
| IgG^{b} | | | | | | |
| MAG1_ | 64 | 78 | 69 | 74 | 2.91 | 0.46 |
| 5.2 or | (44~81) | (62~90) | (53~82) | (63~83) | (1.5~5.8) | (.3~.8) |
| third | | | | | | |
| tertile | | | | | | |
| of | | | | | | |
| Toxo | | | | | | |
| IgG^{b} | | | | | | |
| MAG1_ | 89 | 68 | 68 | 89 | 2.79 | 0.16 |
| 4.2 or | (72~98) | (50~82) | (56~77) | (74~96) | (1.7~4.5) | (.05~.5) |
| MAG1_ | | | | | | |
| 5.2 or | | | | | | |
| third | | | | | | |
| tertile | | | | | | |
| of | | | | | | |
| Toxo | | | | | | |
| IgG^{b} | | | | | | |

Abbreviations: IgG, immunoglobulin G; LR+, positive likelihood ratio; LR−, negative likelihood ratio; MAG1, matrix antigen 1; NPV, negative predictive value; PPV, positive predictive value; Toxo, Toxoplasma.
^{a}Number ( ): % (95% confidence interval).
^{b}Toxoplasma IgG between 3.22 and 4.00 optical density (OD) units.

TABLE 5
Distribution of Toxoplasma Serotype in Relation to Toxoplasmic
Encephalitis

| | Case | | Control | | | | Fisher |
|---|---|---|---|---|---|---|---|
| | (n = 28) | | (n = 37) | | Total | | Exact Test |
| Serotype | n | % | n | % | n | % | P |
| I | 0 | 0.00 | 3 | 8.11 | 3 | 4.62 | .167 |
| II | 9 | 32.14 | 8 | 21.62 | 17 | 26.15 | |
| III | 1 | 3.57 | 0 | 0.00 | 1 | 1.54 | |
| Atypical | 3 | 10.71 | 1 | 2.70 | 4 | 6.15 | |
| Indeterminate | 15 | 53.57 | 25 | 67.57 | 40 | 61.54 | |

### References in Example 1

1. Luft B J, Remington J S. Toxoplasmic encephalitis in AIDS. Clin Infect Dis 1992; 15:211-22.
2. Xiao J, Yolken R H. Strain hypothesis of Toxoplasma gondii infection on the outcome of human diseases. Acta Physiol (Oxf) 2015; 213:828-45.
3. Suzuki Y, Wong S Y, Grumet F C, et al. Evidence for genetic regulation of susceptibility to toxoplasmic encephalitis in AIDS patients. J Infect Dis 1996; 173:265-8.
4. Habegger de Sorrentino A, López R, Motta P, et al. HLA class II involvement in HIV-associated toxoplasmic encephalitis development. Clin Immunol 2005; 115:133-7.
5. Belanger F, Derouin F, Grangeot-Keros L, Meyer L. Incidence and risk factors of toxoplasmosis in a cohort of human immunodeficiency virus-infected patients: 1988-1995. HEMOCO and SEROCO Study Groups. Clin Infect Dis 1999; 28:575-81.
6. Derouin F, Leport C, Pueyo S, et al. Predictive value of Toxoplasma gondii antibody titres on the occurrence of toxoplasmic encephalitis in HIV-infected patients. ANRS 005/ACTG 154 Trial Group. AIDS 1996; 10:1521-7.
7. Hellerbrand C, Goebel F D, Disko R. High predictive value of Toxoplasma gondii IgG antibody levels in HIV-infected patients for diagnosis of cerebral toxoplas-mosis. Eur J Clin Microbiol Infect Dis 1996; 15:869-72.
8. Xiao J, Viscidi R P, Kannan G, et al. The Toxoplasma MAG1 peptides induce sex-based humoral immune response in mice and distinguish active from chronic human infection. Microbes Infect 2013; 15:74-83.
9. Xiao J, Li Y, Prandovszky E, et al. Behavioral abnormalities in a mouse model of chronic toxoplasmosis are associated with MAG1 antibody levels and cyst burden. PLoS Negl Trop Dis 2016; 10:e0004674.
10. Detels R, Jacobson L, Margolick J, et al. The Multicenter AIDS Cohort Study, 1983 to. Public Health 2012; 126:196-8.
11. Kaslow R A, Ostrow D G, Detels R, Phair J P, Polk B F, Rinaldo C R Jr. The Multicenter AIDS Cohort Study: rationale, organization, and selected characteristics of the participants. Am J Epidemiol 1987; 126:310-8.
12. Israelski D M, Chmiel J S, Poggensee L, Phair J P, Remington J S. Prevalence of Toxoplasma infection in a cohort of homosexual men at risk of AIDS and toxoplasmic encephalitis. J Acquir Immune Defic Syndr (1988) 1993; 6:414-8.
13. Xiao J, Buka S L, Cannon T D, et al. Serological pattern consistent with infection with type I Toxoplasma gondii in mothers and risk of psychosis among adult offspring. Microbes Infect 2009; 11:1011-8.
14. Ajzenberg D, Lamaury I, Demar M, et al. Performance testing of PCR assay in blood samples for the diagnosis of toxoplasmic encephalitis in AIDS patients from the French Departments of America and Genetic Diversity of Toxoplasma gondii: a prospective and multicentric study. PLoS Negl Trop Dis 2016; 10:e0004790.
15. Alfonso Y, Fraga J, Fonseca C, et al. Molecular diagnosis of Toxoplasma gondii infection in cerebrospinal fluid from AIDS patients. Cerebrospinal Fluid Res 2009; 6:2.
16. Matinella A, Lanzafame M, Bonometti M A, et al. Neurological complications of HIV infection in pre-HAART and HAART era: a retrospective study. J Neurol 2015; 262:1317-27.
17. Kodym P, Malý M, Beran O, et al. Incidence, immunological and clinical characteristics of reactivation of latent Toxoplasma gondii infection in HIV-infected patients. Epidemiol Infect 2015; 143:600-7.
18. Nissapatorn V. Toxoplasma gondii and HIV: a never-ending story. Lancet HIV 2017; 4:e146-7.
19. Egger M, May M, Chêne G, et al; ART Cohort Collaboration. Prognosis of HIV-1-infected patients starting highly active antiretroviral therapy: a collaborative analysis of prospective studies. Lancet 2002; 360:119-29.
20. Ford N, Shubber Z, Meintjes G, et al. Causes of hospital admission among people living with HIV worldwide: a systematic review and meta-analysis. Lancet HIV 2015; 2:e438-44.
21. Antinori A, Larussa D, Cingolani A, et al; Italian Registry Investigative NeuroAIDS. Prevalence, associated factors, and prognostic determinants of AIDS-related toxoplasmic encephalitis in the era of advanced highly active anti-retroviral therapy. Clin Infect Dis 2004; 39:1681-91.
22. Vaughan L B, Wenzel R P. Disseminated toxoplasmosis presenting as septic shock five weeks after renal transplantation. Transpl Infect Dis 2013; 15:E20-4.
23. Derouin F, Pelloux H; ESCMID Study Group on Clinical Parasitology. Prevention of toxoplasmosis in transplant patients. Clin Microbiol Infect 2008; 14:1089-101.
24. Robert-Gangneux F, Meroni V, Dupont D, et al. Toxoplasmosis in transplant recipients, Europe, 2010-2014. Emerg Infect Dis 2018; 24:1497-504.
25. Glasner P D, Silveira C, Kruszon-Moran D, et al. An unusually high prevalence of ocular toxoplasmosis in southern Brazil. Am J Ophthalmol 1992; 114:136-44.
26. Desmonts G, Couvreur J, Thulliez P. Congenital toxoplasmosis: 5 cases of mother-to-child transmission of pre-pregnancy infection. Presse Med 1990; 19:1445-9.
27. Marty P, Bongain A, Rahal A, et al. Prenatal diagnosis of severe fetal toxoplas-mosis as a result of toxoplasmic reactivation in an HIV-1 seropositive woman. Prenat Diagn 1994; 14:414-5.
28. Minkoff H, Remington J S, Holman S, Ramirez R, Goodwin S, Landesman S. Vertical transmission of Toxoplasma by human immunodeficiency virus-infected women. Am J Obstet Gynecol 1997; 176:555-9.
29. Silveira C, Ferreira R, Muccioli C, Nussenblatt R, Belfort R Jr. Toxoplasmosis transmitted to a newborn from the mother infected 20 years earlier. Am J Ophthalmol 2003; 136:370-1

### Example 2: Monitoring MAG1 Antibody Levels to Assess Anti-PD-L1 Treatment for Neuroinflammation Caused by Chronic Toxoplasma Infection

Toxoplasma gondii can infect the host brain and trigger neuroinflammation. Such neuroinflammation might persist for years if the infection is not resolved, resulting in harmful outcomes for the brain. We have previously demonstrated the efficacy of immunotherapy targeting the PD-1 pathway on clearance of Toxoplasma tissue cysts.

We aimed to test whether parasite clearance would lead to the resolution of neuroinflammation in infected brains. We established chronic Toxoplasma infection in BALB/c mice using the cyst-forming Prugniaud strain. Mice then received αPD-L1 or isotype control antibodies. After completion of the therapy, mice were euthanized six weeks later. The number of brain tissue cysts, Toxoplasma-specific CD8+ T cell proliferation and IFN-γ secretion, serum cytokine and chemokine levels, and CNS inflammation were measured.

In αPD-L1-treated mice, we observed reduced brain tissue cysts, increased spleen weight, elevated IFN-α production by antigen-specific CD8+ T cells, and a general increase in multiple serum cytokines and chemokines. Importantly, αPD-L1-treated mice displayed attenuation of meningeal lymphocytes and astrocyte and C1q activation. The reduction in inflammation-related proteins is correlated with reduced parasite burden. These results suggest that promoting systemic immunity results in parasite clearance, which in turn alleviates neuroinflammation. Our study may have implications for some brain infections where neuroinflammation is a critical component.

Toxoplasma gondii can infect the brain and trigger neuroinflammation. Inflammatory response includes activation of microglia and astrocyte, upregulation of complement components, infiltration of immune cells, and elevation of cytokines and chemokines [1-4]. Although these inflammatory processes effectively control parasite reactivation, the state of neuroinflammation may persist for years if the infection does not resolve. Persistent inflammation has been associated with multiple neuropathologies in Toxoplasma-infected animals. Studies have reported cortical neurodegeneration [5] and changes in neuronal morphology [6], brain connectivity [7], and neurotransmitter pathways [8]. Neuroinflammation is a critical component of virtually all neurodegenerative diseases. Not surprisingly, Toxoplasma seroprevalence in humans has been considered a risk factor for various brain disorders such as schizophrenia, Alzheimer's, and Parkinson's disease [9].

Toxoplasma tissue cysts are reported to significantly affect inflammatory responses [1, 5, 10-12]. As revealed by transcriptome analysis of host brains, expression of many immune- and neuronal-related genes was mainly associated with cyst burden. Tanaka et al. [10] found positive correlations between the number of parasites in the infected mouse brains and the expression levels of genes involved in host immune responses. In contrast, genes that had a negative correlation with parasite numbers were those genes that are predicted to be involved in neurological functions, such as small-GTPase-mediated signal transduction and vesicle-mediated transport. Boillat et al. [11] observed a link between cyst burden and markers associated with pro-and anti-inflammatory cascades, astrocyte activation, neuronal loss, and downregulation of neurotransmitter pathways. The plasma level of proinflammatory cytokines, such as IFN-γ and IL-12/IL-23p40, have also been associated with cyst burden. We have observed that parasite burden is a critical determinant for expressions of several inflammatory molecules (CCL5, IL-12p70, IL-12p40, and TNF-α) and markers for synaptic remodeling (C1q) and neuronal cell damage (FJB) [1, 5, 12]. Such changes were not present in mice with lower cyst burden or mice that did not progress to the chronic stage of infections (cyst free).

Neuroinflammation is defined as an inflammatory response within the brain or spinal cord, with signals originating from barrier tissues such as brain vasculature, surrounding meninges, and the choroid plexus [13-15]. The meninges enclose the brain and spinal cord, which have traditionally been considered structures that protect the brain parenchyma. However, recent studies suggest that the meninges are an immunologically active compartment communicating with the periphery via the meningeal lymphatic system [16]. Increasingly studies demonstrate that meninges play a critical role in the maintenance of brain function and CNS disease [17-19]. Walker-Caulfield et al. [20 ] reported that meningeal inflammation precedes inflammation in the CNS and parallels remittances and relapses in a murine model of multiple sclerosis.

Studies have shown that diminishing the inflammatory responses rescues Toxoplasma-associated neuropathogenesis and behavioral abnormalities [21, 22]. Although the treatments used in these studies may not eliminate tissue cysts, they significantly reduced the expression of inflammatory mediators in the brains of infected mice. As described above, cyst burden is a primary determinant for neuroinflammation. We hypothesized that diminishing neuroinflammation by parasite clearance is superior to anti-inflammatory drugs. Currently, available anti-Toxoplasma drugs are ineffective against tissue cysts. We have previously demonstrated that blockade of the PD-1/PD-L1 pathway significantly reduces the number of Toxoplasma tissue cysts in the brain [23]. Many studies have shown the efficacy of PD-L1 blockade in reversing T-cell exhaustion [24].

We tested whether parasite clearance via immunotherapy targeting the PD-1 pathway would attenuate brain inflammation. We previously used a model of virulent Toxoplasma strain in CD1 outbred mice [23]. This model resembles the heterogeneous effects of Toxoplasma infection in humans. However, the model generates a limited number of tissue cysts (˜200) because virulent strains do not readily develop tissue cysts in these mice. A limitation is the lack of well-defined T-cell epitopes elicited by Toxoplasma in CD-1 mice, making the enumeration of antigen-specific T-cell responses difficult. Here we used inbred BALB/c mice susceptible to a cyst-competent Toxoplasma strain and amenable to immunological studies. Specific objectives included: 1) will the treatment apply to a different model of Toxoplasma infection? 2) will the treatment reverse the phenotype of exhausted antigen-specific CD8+ T cells? 3) will the treatment reduce neuroinflammation?

### Methods

### Mouse Model of Chronic Toxoplasma Infection

We established chronic infection of BALB/c mice with Toxoplasma prugniaud (Pru) strain. BALB/c mice were chosen because three Toxoplasma-derived CD8+ T cell epitopes mediating Ld-restricted protective immunity have been identified in the BALB/c strain [25, 26]. The Pru strain is cyst-competent [27]. Briefly, 9-week-old male BALB/c mice (The Jackson Laboratory) were infected intraperitoneally (i.p.) with 400 tachyzoites of Pru strain. Control mice received vehicle only (phosphate-buffered saline [PBS]). Male mice were infected because our previous study showed males generate more tissue cysts than females [28].

Infection was confirmed serologically by the presence of IgG antibodies to the whole Toxoplasma organism and peptide antigens of the Toxoplasma cyst protein MAG1. The anti-Toxoplasma antibodies were measured using a modified commercial ELISA kit [12], and anti-MAG1 antibodies were determined using a previously developed MAG1 ELISA assay [29]. Serum was diluted at 1:100 for antibody testing.

### Mice Grouping

At six weeks post-infection (wpi) when the maximum number of tissue cysts is supposed to reach the brain, blood was collected from the tail vein of the mice, and sera were isolated. Toxoplasma IgG and MAG1 antibodies were measured. Mice were then assigned to three groups, infected (n=5), αPD-L1 (n=7), and isotype control (n=5), stratified by serum MAG1 level. MAG1 antibody is a serological marker for brain tissue cysts [12], which level varies by the number of cysts in the brain. The stratification ensured cyst burden would not differ in the three groups before treatment. The group of mice that were mock-infected with PBS (n=5) served as uninfected controls.

### Reagents

Antigen and peptide selection. Three Toxoplasma-derived Ld-restricted CD8+ T cell epitopes, the GRA6-derived HF10 (HPGSVNEFDF (SEQ ID NO: 3)) [25], the ROP7-derived IF9 (IPAAAGRFF (SEQ ID NO: 4)), and the GRA4-derived SM9 (SPMNGGYYM (SEQ ID NO: 5)) peptides [26], were synthesized by GenScript with high purity (>95%).

H-2Ld HF10 (HPGSVNEFDF (SEQ ID NO: 3)) tetramers for detection of GRA6-specific CD8 cells were obtained from the NIH Tetramer Core Facility.

### In Vivo Blockade of the PD-1 Pathway

Anti-PD-L1 antibodies were administrated to infected mice at 6 wpi. For blockade of the PD-1 pathway, mice received via i.p. 200 μg of rat antimouse PD-L1 antibody (10F.9G2, Biolegend) every 3 days for 2 weeks. Mice that received IgG2b antibody served as isotype control. The ability of this anti-PD-L1 antibody regimen to block the PD-1 pathway has been previously demonstrated [23]. Mice were sacrificed six weeks following the final injection, and brains, spleens, and blood were collected.

Cell preparation and Flow Cytometry

Spleen cells from mice were collected by splenic grinding, each step with 5 mL RPMI-1640 (Gibco). Cells were subsequently washed twice and then resuspended in RPMI-1640. For peptide stimulation, cells were incubated overnight with individual peptides or a mixture of the 3 peptides in the presence of Brefeldin A. Activation cocktail (Biolegend) was used as a positive control. Background stimulation was assessed by stimulation with PBS control. Antigen-specific CD8+ T cells were analyzed by staining for CD3 (clone 17A2), CD8 (clone 53-6.7), and intracellular IFN-γ (clone XMG1.2) using a flow cytometer in the Johns Hopkins flow cytometry core. Data were acquired and analyzed using BD FACSDiva™ software.

### Serum Cytokine Quantification

When mice were sacrificed, blood was collected and serum isolated. Levels of 31 cytokines and chemokines in the serum were measured using Bio-Plex multiplex assay (Bio-Rad) including: BCA-1/CXCL13, IL-4, MIP-1α/CCL3, CTACK/CCL27, IL-6, MIP-1β/CCL4, ENA-78/CXCL5, IL-10, MIP-3α/CCL20, Eotaxin/CCL11, IL-16, RANTES/CCL5, Eotaxin-2/CCL24, IP-10/CXCL10, MIP-3β/CCL19, Fractalkine/CX3CL1, I-TAC/CXCL11, SCYB16/CXCL16, GM-CSF, KC/CXCL1, SDF-1α/CXCL12, I-309/CCL1, MCP-1/CCL2, TARC/CCL17, IFN-γ, MCP-3/CCL7, TNF-α, IL-1β, MCP-5/CCL12, IL-2, MDC/CCL22. All samples were analyzed according to the manufacturer's instructions.

### Cyst Enumeration

The brain was cut sagittally along the midline. One-half of the brain was used for the preparation of homogenates, while the other half served for immunohistochemical analyses. Tissue cyst was enumerated in brain homogenates as described previously [12]. Briefly, samples were examined using a fluorescent microscope, and the number of brain cysts was counted in seven samples of 8-μl suspension per brain homogenate. All numbers reported correspond to the numbers obtained for the half-brain multiplied by 2.

### Meninges Collection

Meninges were collected as described by Louveau et al. [30]. Briefly, the skull was isolated, and the inferior jaw, lower orbits, and nasal bone were removed. The skullcap was obtained by cutting the post-tympanic hook and placing it onto a petri dish with ice-cold PBS. Under a dissecting binocular, the meninges were harvested with forceps starting at the level of the olfactory lobe. The tissues were then snap-frozen.

### Staining

The whole meninges were mounted and stained with DAPI (4′,6-diamidino-2-phenylindole; Sigma-Aldrich), rat anti-mouse anti-CD3 antibody (monoclonal, eBioscience, cat. 14-0032-85, dilution 1:1000), and anti-mouse anti-CD14 antibody (monoclonal, cat. 11-0141-82, 1:50, eBioscience). Images were visualized using a confocal laser microscope (Zeiss LSM700).

### Immunoblot Analyses

Total protein was extracted from brain homogenates using T-PER™ Tissue Protein Extraction Reagent (Thermo Scientific) added with Halt Protease and Phosphatase Inhibitor Cocktail (Thermo Scientific). Protein concentrations were measured using a BCA protein assay kit (Thermo Scientific). Total protein (10˜40 μg) was loaded on 4-20% TGX protein gel (Bio-Rad) for electrophoresis under non-reducing or reducing conditions and transferred to PVDF membranes (Bio-Rad). The membranes were blocked with Starting Block T20 (TBS) Blocking Buffer (Thermo Scientific) for 1 h at room temperature, followed by incubation with primary antibodies at 4° C. overnight. Proteins were probed with primary antibodies for IBA1 (polyclonal, cat. 016-20001, 1:400, Wako), GFAP (polyclonal, cat. ab7260, 1:500, Abcam), C1q (monoclonal, cat. ab71089, 1:1000, Abcam), and C4 (monoclonal, cat. NB200-541, 1:10, Novus Biologicals). Bands were visualized using enhanced chemiluminescence (SuperSignal West Femto Maximum Sensitivity Substrate, Thermo Scientific). Protein values were normalized for corresponding values of β-actin. Relative optical density was determined using ImageLab software (Bio-Rad).

### Statistical Analysis

The significant differences between the αPD-L1 and isotype control groups were analyzed by Student's t-test. For cytokine analysis, we then performed multiple testing corrections with a false discovery rate (FDR) set at 5%, in addition to a 1.5-fold change threshold, to detect upregulated molecules between the two groups. For multiple groups, ANOVA with Bonferroni's multiple comparisons was applied. Paired t-test was used to compare the mean ODs of MAG1 antibodies before and after treatment. Correlation analysis was performed using Pearson's correlation coefficient (r). Data are presented as means±SEM. Statistical analyses were conducted in Graph-Pad Prism V9.2.0. Significance was denoted as a P of <0.05.

### Results

Toxoplasma infection generates a variable number of tissue cysts in the brains of BALB/c mice.

High levels of Toxoplasma IgG antibody were found in all exposed mice (OD=4.0±0). However, levels of MAG1 antibody varied greatly among these mice (OD=0.88±0.11), suggesting individual mice have different parasite burdens. Mice were assigned to three groups stratified by serum MAG1 level, with levels in the infected group ranging from 0.30 to 1.16 (mean OD=0.80±0.15), in the αPD-L1 group ranging from 0.44 to 1.26 (OD=0.81±0.12), and in the isotype control ranging from 0.39 to 1.51 (OD=0.86±0.20). There were no significant differences in MAG1 levels among the three groups (p=0.91).

### PD-L1 Blockade Effectively Reduces Brain Tissue Cysts

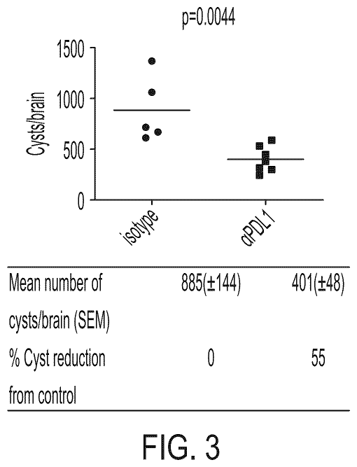

In our previous study [23], we show tissue cyst reduction occurs two weeks following the completion of the PD-L1 injection. Here, the mice were euthanized six weeks post-treatment because we hypothesized that neuroinflammation resolution would be a slower process than parasite clearance. We found a significantly lower number of brain tissue cysts in αPD-L1-treated mice than in isotype-treated (mean: 401 vs 885, p=0.0044, FIG. 3), demonstrating the therapeutic efficacy of the PD-L1 blockade in parasite control (55% reduction). To determine if reactivation from bradyzoite to tachyzoite occurs, we measured the tachyzoite-specific SAG1 gene. No SAG1 gene expression was detected in the brain tissues of both groups (data not shown).

### PD-L1 Blockade Induces a Generalized Increase in Serum Cytokine and Chemokine Levels

We investigated whether PD-L1 blockade affects the serum cytokine and chemokine levels. Employing Bio-Plex multiplex assay, we quantified 31 serum cytokines and chemokines. An increase in all cytokines and chemokines except CXCL16 was observed in αPD-L1-treated mice (Supplementary Excel spreadsheet 1). Using the cut-off criteria for multiple comparisons described in Statistical analysis (5% FDR, 1.5 fold change), we found that 13 of 30 cytokines and chemokines were significantly elevated. The increased expression occurred in both pro- and anti-inflammatory cytokines involving IFN-γ, IL-1β, IL-2, IL-4, IL-10, IL-16, and TNF-α. The increased expression of chemokines included the T cell chemoattractants CCL5, CXCL11, CCL27 and several leukocytes chemoattractant CX3CL1, CCL20, and CCL22. Among these molecules, CCL27 and IL-10 showed the highest fold change, 41 and 25-fold, respectively.

### PD-L1 Blockade Diminishes Meningeal Lymphocytes

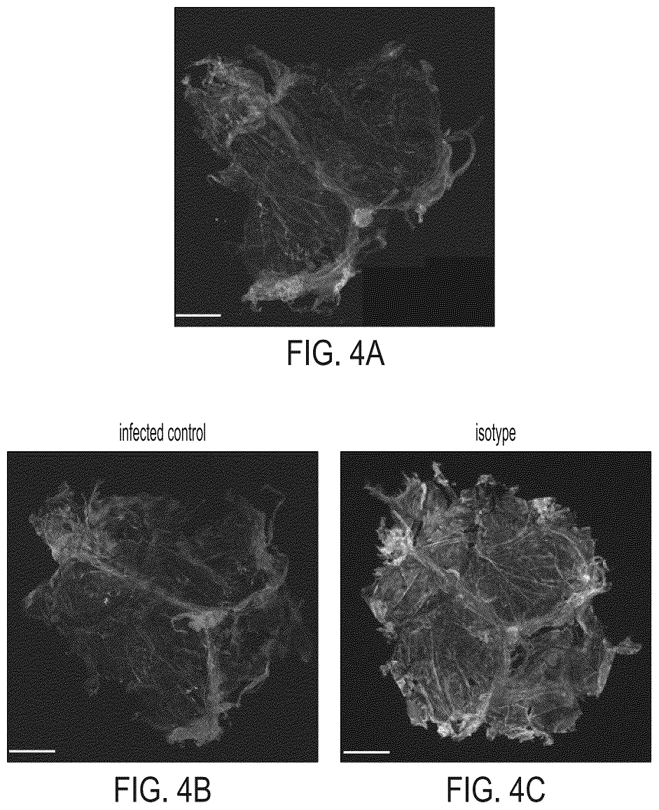

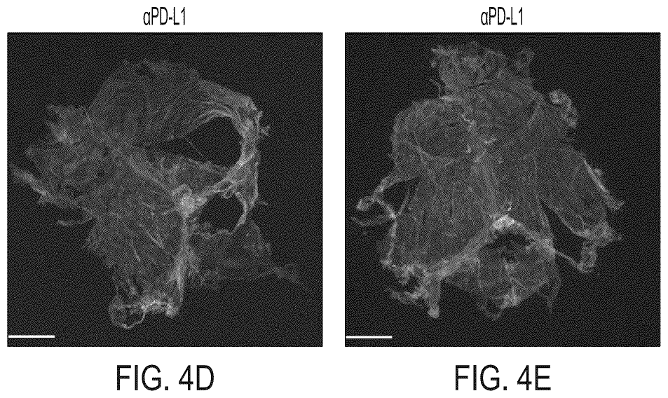

The meningeal compartment is a direct route for immune-related communication between the central nervous system and the peripheral immune system [19]. The whole mount of mouse brain meninges was dissected and stained for CD3e (T cells), CD14 (monocytes), and DAPI (nuclei). Labeling of these cells in uninfected mice revealed a baseline level of expression (FIG. 4A). Toxoplasma infection induced an accumulation of CD3+ T lymphocytes in the meninges, with a high concentration of cells found close to the dural sinuses, as shown in both infected and isotype-treated mice (FIGS. 4B and 4C). Moreover, CD14 staining was more pronounced throughout the meningeal compartments in infected and isotype-treated mice (FIGS. 4B and 4C). Interestingly, the staining intensity of CD3 and CD14 were decreased in αPD-L1-treated mice (FIGS. 4D and 4E). These results suggested that T lymphocytes and monocytes were reduced in the meninges six weeks after the completion of PD-L1 treatment.

### PD-L1 Blockade Suppresses Astrocyte and Complement Activation

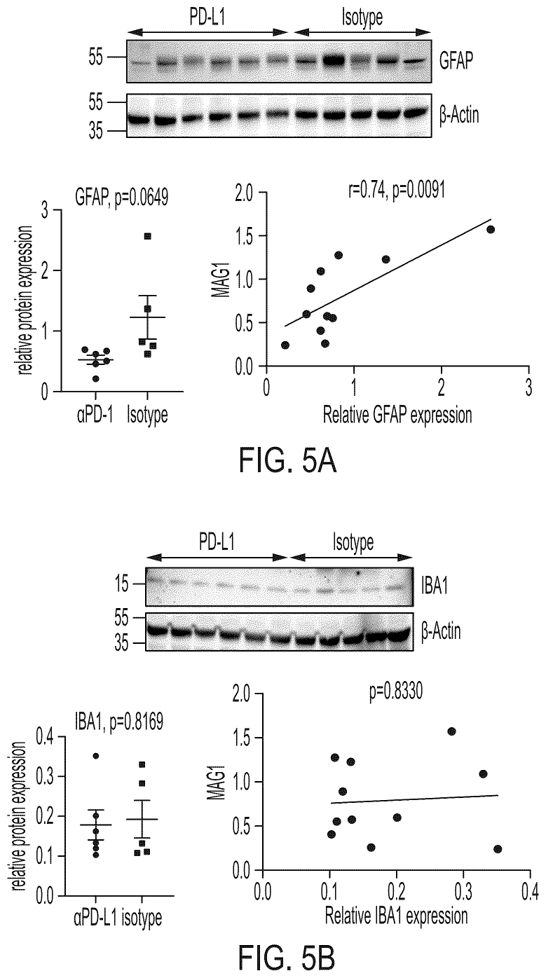

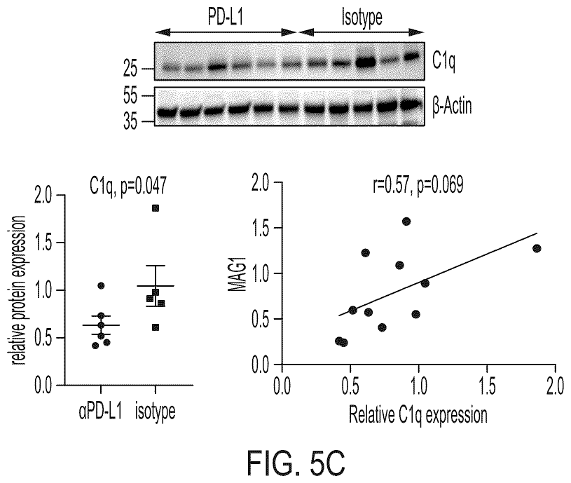

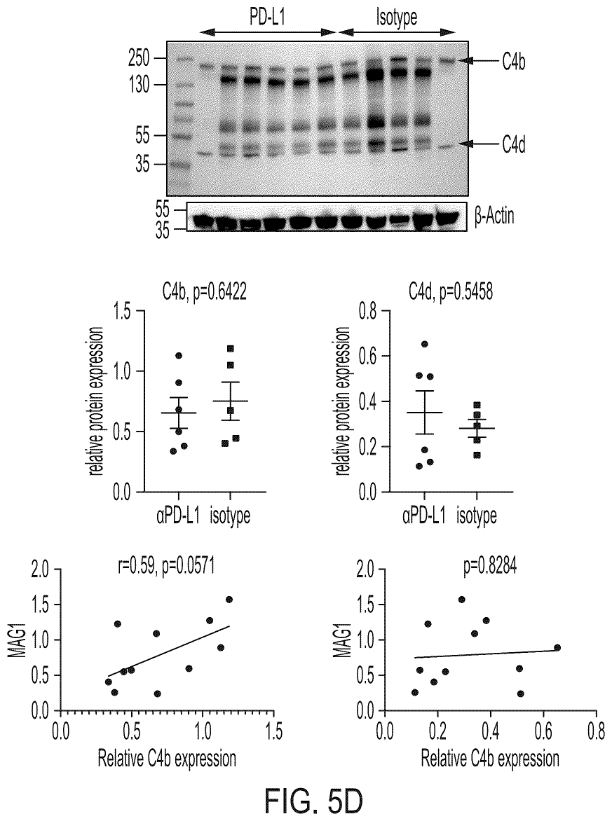

We examined the extent of the brain inflammatory response since meningeal lymphatic vessels communicate with the brain parenchyma [16]. The primary CNS-resident cells involved in inflammation are astrocytes and microglia. GFAP and IBA-1 are well-recognized markers of activated astrocytes and microglia, respectively. We previously identified the activation of cerebral complement components in chronic Toxoplasma infection [1, 5]. We thus compared the expression of these markers in brain homogenates of mice that received αPD-L1 or isotype control. Western blots showed that the expression of GFAP and C1q in αPD-L1-treated mice was lower than that in isotype-treated mice (GFAP, mean: 0.527 vs 1.227, p=0.0649; C1q, mean: 0.633 vs 1.045, p=0.047; FIGS. 5A and 5C). However, the expression of IBA1 and C4b/d did not differ between the two groups (Ps>0.5, FIGS. 7B and 7D). Under the non-reducing condition, immunoblot with anti-C4b/d antibody revealed an approximately 200 kDa band corresponding to the molecular weight of mouse C4b and a ˜45 kDa band corresponding to the molecular weight of C4d (FIG. 5D). MAG1 antibody levels measured after treatment correlated with the levels of GFAP (r=0.74, p=0.0091), and a trend towards significant correlation with C1q (r=0.57, p=0.069) and C4b (r=0.59, p=0.0571).

### Cyst Reduction Affects the MAG1 Antibody Level

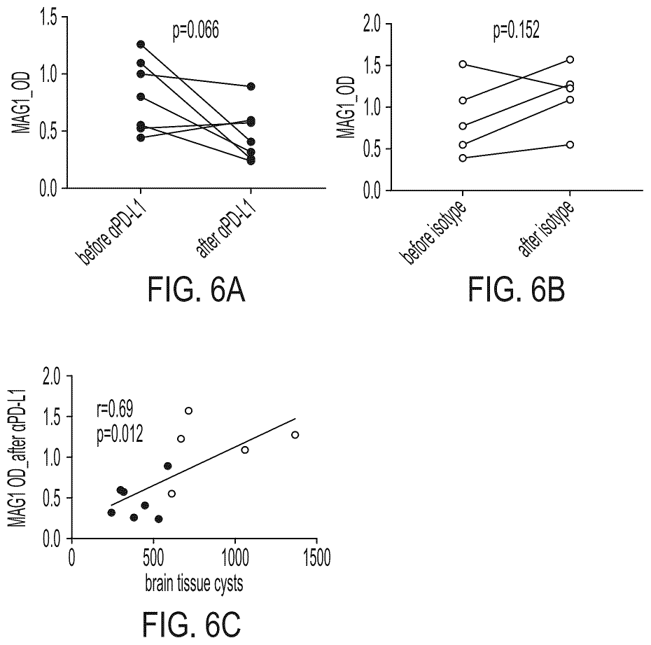

At the start of treatment, the αPD-L1 and isotype control mice by design had comparable levels of MAG1 antibody, as noted above. Since the cyst burden at the end of treatment was lower in αPD-L1-treated mice than in isotype control mice, we determined whether the antibody levels changed over the treatment. As shown in FIG. 6A, MAG1 antibody levels decreased six weeks following the PD-L1 treatment (mean OD from 0.81 to 0.46), although the changes were highly variable, from negative to a decrease of 0.854 OD units. The decline showed a trend toward significance (Paired t-test, p=0.0662). In contrast, MAG1 antibody levels showed a general increase in the isotype control group (mean OD from 0.86 to 1.14, FIG. 6B), although not significant (p=0.1518).

A correlation between change in MAG1 antibody and reduction of cyst burden cannot be directly demonstrated because cyst burden is only inferred before treatment, based on MAG1 antibody level. Direct measurement of brain cysts requires that mice be euthanized. However, we found, as expected from our previous studies [12], a positive correlation between MAG1 antibody level and the number of brain cysts at the termination of the experiment across both αPD-L1 and isotype control groups (r=0.69, p=0.012, FIG. 6C). These findings provide indirect support for a dynamic relationship between MAG1 antibody levels and cyst burden.

### Discussion

In mice with chronic Toxoplasma infection, we examined the effects of PD-L1 blockade on parasite control, the rescue of exhausted antigen-specific CD8+ T cells, and reversal of neuroinflammation. Consistent with our previous study [23], this immunotherapy significantly reduced the number of brain tissue cysts. Although the frequency of antigen-specific CD8+ T cells was not affected by PD-L1 blockade, the CD8+ cells produced more IFN-γ. There was a marked increase in serum levels of cytokines and chemokines in αPD-L1-treated mice. While systemic immune responses were enhanced, CNS neuroinflammation was reduced, as evidenced by attenuation of meningeal inflammatory responses and astrocyte and C1q activation. The reduction in the inflammatory response is correlated with a reduced parasite burden. Our results suggest that the inflammatory response is a consequence of persistent parasite burden and can be reversed, at least partially, by diminishing the parasite burden. Neuroinflammation is a prime component of virtually all neurodegenerative diseases [34]. However, anti-inflammatory and immunosuppressive therapies have performed poorly in clinical trials [35]. Our study provides a proof of concept for the resolution of neuroinflammation via PD-L1 blockade in models of brain infection.

Consistent with our previous study [23], we observed a marked increase in levels of multiple serum cytokines and chemokines in αPD-L1-treated mice. These biomarkers are produced by both immune and non-immune cells that can interact with each other and elicit biological activity. Elevation of serum pro- and anti-inflammatory cytokines, such as IFN-γ, IL-1β, IL-2, IL-4, IL-10, IL-16, and TNF-α, might be a hallmark of the functional cure of Toxoplasma-infected mice. Elevation of many different types of chemokines (CCL5, CXCL11, CCL27, CX3CL1, CCL20, and CCL22) demonstrated that PD-L1 blockade activates cross-talk between T cells, macrophages, and dendritic cells. It is tempting to speculate that the coordinated activation of innate and adaptive immune cells is key to successful anti-Toxoplasma responses for αPD-L1 immunotherapy. A primary distinction between exhausted and functional CD8+ T cells is the ability to produce cytokines such as IFN-γ, TNF-α, and IL-2 upon T cell receptor stimulation. [40].

We observed decreased inflammatory infiltrates in the meningeal compartment of mice that received the αPD-L1 antibodies 6 weeks following the αPD-L1 treatment. The meninges represent an important barrier and a gateway into the CNS. In line with the decreased meningeal infiltrates, αPD-L1 treated mice had a reduced expression of markers for astrocytes (GFAP) and complement activation (C1q). The results suggest that attenuation of inflammation in meningeal compartments could limit CNS inflammation. The reason, presumably, is due to the reduced parasite burden failing to trigger a robust inflammatory response. Indeed, the reduction in inflammation-related proteins is correlated with reduced parasite burden. Previous reports using animal models of multiple sclerosis shows meningeal inflammation paralleling remittances and relapses; infiltrates and inflammatory mediators decrease during remission [13, 20]. We previously observed a rapid mobilization of leukocytes into brains via CSF-filled compartments 2 weeks following the PD-L1 blockade [23]. Timing differences between completion of the treatment and euthanasia (short vs long periods) may account for the distinct findings in meningeal compartments. Parasite clearance can occur rapidly through peripheral immune cell infiltration, but the neuroinflammation triggered by tissue cysts may require time to diminish.

We have previously shown that MAG1 antibodies are a serological marker of cyst burden in the brain [12]. Consistent with the clearance of brain tissue cysts, MAG1 antibody levels declined in mice that received theαPD-L1. Moreover, the number of tissue cysts detected at the end of the experiment correlated with MAG1 antibody levels measured post-treatment. The results suggest that MAG1 antibodies might serve as a biomarker for monitoring the efficacy of anti-Toxoplasma agents directed at chronic infection. Moreover, the correlation between MAG1 antibodies and the expression of some inflammation-related proteins suggests that MAG1 antibodies could indicate neuroinflammation triggered by Toxoplasma's brain-dwelling.

### References in Example 2

1. Xiao J, Li Y, Gressitt K L, He H, Kannan G, Schultz T L, Svezhova N, Carruthers V B, Pletnikov M V, Yolken R H, Severance E G: Cerebral complement C1q activation in chronic Toxoplasma infection. Brain Behav Immun 2016, 58:52-56.
2. Hermes G, Ajioka J W, Kelly K A, Mui E, Roberts F, Kasza K, Mayr T, Kirisits M J, Wollmann R, Ferguson D J, et al: Neurological and behavioral abnormalities, ventricular dilatation, altered cellular functions, inflammation, and neuronal injury in brains of mice due to common, persistent, parasitic infection. J Neuroinflammation 2008, 5:48.
3. Landrith T A, Harris T H, Wilson E H: Characteristics and critical function of CD8+ T cells in the Toxoplasma-infected brain. Semin Immunopathol 2015, 37:261-270.
4. Xiao J: Behavioral Changes Induced by Latent Toxoplasmosis Could Arise from CNS Inflammation and Neuropathogenesis. Curr Top Behav Neurosci 2022.
5. Li Y, Severance E G, Viscidi R P, Yolken R H, Xiao J: Persistent Toxoplasma Infection of the Brain Induced Neurodegeneration Associated with Activation of Complement and Microglia. Infect Immun 2019, 87.
6. David C N, Frias E S, Szu J I, Vieira P A, Hubbard J A, Lovelace J, Michael M, Worth D, McGovern K E, Ethell I M, et al: GLT-1-Dependent Disruption of CNS Glutamate Homeostasis and Neuronal Function by the Protozoan Parasite Toxoplasma gondii. PLoS Pathog 2016, 12:e1005643.
7. Parlog A, Harsan L A, Zagrebelsky M, Weller M, von Elverfeldt D, Mawrin C, Korte M, Dunay I R: Chronic murine toxoplasmosis is defined by subtle changes in neuronal connectivity. Dis Model Mech 2014, 7:459-469.
8. Brooks J M, Carrillo G L, Su J, Lindsay D S, Fox M A, Blader I J: Toxoplasma gondii Infections Alter GABAergic Synapses and Signaling in the Central Nervous System. mBio 2015, 6:e01428-01415.
9. Xiao J, Prandovszky E, Kannan G, Pletnikov M V, Dickerson F, Severance E G, Yolken R H: Toxoplasma gondii: Biological Parameters of the Connection to Schizophrenia. Schizophr Bull 2018, 44:983-992.
10. Tanaka S, Nishimura M, Ihara F, Yamagishi J, Suzuki Y, Nishikawa Y: Transcriptome analysis of mouse brain infected with Toxoplasma gondii. Infect Immun 2013, 81:3609-3619.
11. Boillat M, Hammoudi P M, Dogga S K, Pages S, Goubran M, Rodriguez I, Soldati-Favre D: Neuroinflammation-Associated Aspecific Manipulation of Mouse Predator Fear by Toxoplasma gondii. Cell Rep 2020, 30:320-334 e326.
12. Xiao J, Li Y, Prandovszky E, Kannan G, Viscidi R P, Pletnikov M V, Yolken R H: Behavioral Abnormalities in a Mouse Model of Chronic Toxoplasmosis Are Associated with MAG1 Antibody Levels and Cyst Burden. PLoS Negl Trop Dis 2016, 10:e0004674.
13. Russi A E, Brown M A: The meninges: new therapeutic targets for multiple sclerosis. Transl Res 2015, 165:255-269.
14. Zlokovic B V: Neurovascular pathways to neurodegeneration in Alzheimer's disease and other disorders. Nat Rev Neurosci 2011, 12:723-738.
15. Schwartz M, Baruch K: The resolution of neuroinflammation in neurodegeneration: leukocyte recruitment via the choroid plexus. EMBO J 2014, 33:7-22.
16. Louveau A, Smirnov I, Keyes T J, Eccles J D, Rouhani S J, Peske J D, Derecki N C, Castle D, Mandell J W, Lee K S, et al: Structural and functional features of central nervous system lymphatic vessels. Nature 2015, 523:337-341.
17. Alves de Lima K, Rustenhoven J, Kipnis J: Meningeal Immunity and Its Function in Maintenance of the Central Nervous System in Health and Disease. Annu Rev Immunol 2020, 38:597-620.
18. Dragunow M: Meningeal and choroid plexus cells—novel drug targets for CNS disorders. Brain Res 2013, 1501:32-55.
19. Frederick N, Louveau A: Meningeal lymphatics, immunity and neuroinflammation. Curr Opin Neurobiol 2020, 62:41-47.
20. Walker-Caulfield M E, Hatfield J K, Brown M A: Dynamic changes in meningeal inflammation correspond to clinical exacerbations in a murine model of relapsing-remitting multiple sclerosis. J Neuroimmunol 2015, 278:112-122.
21. Lang D, Schott B H, van Ham M, Morton L, Kulikovskaja L, Herrera-Molina R, Pielot R, Klawonn F, Montag D, Jansch L, et al: Chronic Toxoplasma infection is associated with distinct alterations in the synaptic protein composition. J Neuroinflammation 2018, 15:216.
22. Martynowicz J, Augusto L, Wek R C, Boehm S L, 2nd, Sullivan W J, Jr.: Guanabenz Reverses a Key Behavioral Change Caused by Latent Toxoplasmosis in Mice by Reducing Neuroinflammation. mBio 2019, 10.
23. Xiao J, Li Y, Yolken R H, Viscidi R P: PD-1 immune checkpoint blockade promotes brain leukocyte infiltration and diminishes cyst burden in a mouse model of Toxoplasma infection. J Neuroimmunol 2018, 319:55-62.
24. Pauken K E, Wherry E J: Overcoming T cell exhaustion in infection and cancer. Trends Immunol 2015, 36:265-276.
25. Blanchard N, Gonzalez F, Schaeffer M, Joncker N T, Cheng T, Shastri A J, Robey E A, Shastri N: Immunodominant, protective response to the parasite Toxoplasma gondii requires antigen processing in the endoplasmic reticulum. Nat Immunol 2008, 9:937-944.
26. Frickel E M, Sahoo N, Hopp J, Gubbels M J, Craver M P, Knoll L J, Ploegh H L, Grotenbreg G M: Parasite stage-specific recognition of endogenous Toxoplasma gondii-derived CD8+ T cell epitopes. J Infect Dis 2008, 198:1625-1633.
27. Sullivan W J, Jr., Jeffers V: Mechanisms of Toxoplasma gondii persistence and latency. FEMS Microbiol Rev 2012, 36:717-733.
28. Xiao J, Viscidi R P, Kannan G, Pletnikov M V, Li Y, Severance E G, Yolken R H, Delhaes L: The Toxoplasma MAG1 peptides induce sex-based humoral immune response in mice and distinguish active from chronic human infection. Microbes Infect 2013, 15:74-83.
29. Xiao J, Bhondoekhan F, Seaberg E C, Yang O, Stosor V, Margolick J B, Yolken R H, Viscidi R P: Serological Responses to Toxoplasma gondii and Matrix Antigen 1 Predict the Risk of Subsequent Toxoplasmic Encephalitis in People Living With Human Immunodeficiency Virus (HIV). Clin Infect Dis 2021, 73:e2270-e2277.
30. Louveau A, Filiano A J, Kipnis J: Meningeal whole mount preparation and characterization of neural cells by flow cytometry. Curr Protoc Immunol 2018, 121:e50.
31. Chiebao D P, Bartley P M, Chianini F, Black L E, Burrells A, Pena H F J, Soares R M, Innes E A, Katzer F: Early immune responses and parasite tissue distribution in mice experimentally infected with oocysts of either archetypal or non-archetypal genotypes of Toxoplasma gondii. Parasitology 2021, 148:464-476.
32. Strickland G T, Ahmed A, Sell K W: Blastogenic response of Toxoplasma-infected mouse spleen cells to T- and B-cell mitogens. Clin Exp Immunol 1975, 22:167-176.
33. Gigley J P, Bhadra R, Khan I A: CD8 T Cells and Toxoplasma gondii: A New Paradigm. J Parasitol Res 2011, 2011:243796.
34. Frank-Cannon T C, Alto L T, McAlpine F E, Tansey M G: Does neuroinflammation fan the flame in neurodegenerative diseases? Mol Neurodegener 2009, 4:47.
35. Mallah K, Couch C, Borucki D M, Toutonji A, Alshareef M, Tomlinson S: Anti-inflammatory and Neuroprotective Agents in Clinical Trials for CNS Disease and Injury: Where Do We Go From Here? Front Immunol 2020, 11:2021.
36. Bhadra R, Gigley J P, Weiss L M, Khan I A: Control of Toxoplasma reactivation by rescue of dysfunctional CD8+ T-cell response via PD-1-PDL-1 blockade. Proc Natl Acad Sci USA 2011, 108:9196-9201.
37. Radaelli A, Bonduelle O, Beggio P, Mahe B, Pozzi E, Elli V, Paganini M, Zanotto C, De Giuli Morghen C, Combadiere B: Prime-boost immunization with DNA, recombinant fowlpox virus and VLP(SHIV) elicit both neutralizing antibodies and IFNgamma-producing T cells against the HIV-envelope protein in mice that control env-bearing tumour cells. Vaccine 2007, 25:2128-2138.
38. Boyd A, Almeida J R, Darrah P A, Sauce D, Seder R A, Appay V, Gorochov G, Larsen M: Pathogen-Specific T Cell Polyfunctionality Is a Correlate of T Cell Efficacy and Immune Protection. PLoS One 2015, 10:e0128714.
39. Meythaler M, Wang Z, Martinot A, Pryputniewicz S, Kasheta M, McClure H M, O'Neil S P, Kaur A: Early induction of polyfunctional simian immunodeficiency virus (SIV)-specific T lymphocytes and rapid disappearance of SIV from lymph nodes of sooty mangabeys during primary infection. J Immunol 2011, 186:5151-5161.
40. Hope J L, Stairiker C J, Bae E A, Otero D C, Bradley L M: Striking a Balance-Cellular and Molecular Drivers of Memory T Cell Development and Responses to Chronic Stimulation. Front Immunol 2019, 10:1595.
41. Mehta D, Jackson R, Paul G, Shi J, Sabbagh M: Why do trials for Alzheimer's disease drugs keep failing? A discontinued drug perspective for 2010-2015. Expert Opin Investig Drugs 2017, 26:735-739.
42. Cummings J L, Tong G, Ballard C: Treatment Combinations for Alzheimer's Disease: Current and Future Pharmacotherapy Options. J Alzheimers Dis 2019, 67:779-794.
43. Baruch K, Deczkowska A, Rosenzweig N, Tsitsou-Kampeli A, Sharif A M, Matcovitch-Natan O, Kertser A, David E, Amit I, Schwartz M: PD-1 immune checkpoint blockade reduces pathology and improves memory in mouse models of Alzheimer's disease. Nat Med 2016, 22:135-137.
44. Rosenzweig N, Dvir-Szternfeld R, Tsitsou-Kampeli A, Keren-Shaul H, Ben-Yehuda H, Weill-Raynal P, Cahalon L, Kertser A, Baruch K, Amit I, et al: PD-1/PD-L1 checkpoint blockade harnesses monocyte-derived macrophages to combat cognitive impairment in a tauopathy mouse model. Nat Commun 2019, 10:465.

### OTHER EMBODIMENTS

From the foregoing description, it will be apparent that variations and modifications may be made to the invention described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

1. A method of diagnosing Toxoplasma-associated disease in a subject comprising:
obtaining a biological sample from the subject;
culturing the biological sample with a modified Toxoplasma cyst peptide;
measuring the amount of antibodies binding to the modified Toxoplasma cyst peptide; and
diagnosing Toxoplasma-associated disease in the subject.

2. The method of claim 1, wherein the modified Toxoplasma cyst peptide comprises a Toxoplasma matrix antigen 1 (MAG1) peptide comprising one or more amino acid deletions, amino acid substitutions, amino acid insertions or combinations thereof.

3. The method of claim 2, wherein the modified Toxoplasma cyst peptide comprises an amino acid sequence having at least a 50% sequence identity to
(SEQ ID NO: 1)
EKQLRRVEPEHEDNTRVEAR/P/RALLEAKTKELVEPTSKEAEEAR
QILAEQAA.

4. The method of claim 2, wherein the modified Toxoplasma cyst peptide comprises an amino acid sequence having at least a 75% sequence identity to
(SEQ ID NO: 1)
EKQLRRVEPEHEDNTRVEAR/P/RALLEAKTKELVEPTSKEAEEAR
QILAEQAA.

5. The method of claim 2, wherein the modified Toxoplasma cyst peptide comprises an amino acid sequence having at least a 95% sequence identity to
(SEQ ID NO: 1)
EKQLRRVEPEHEDNTRVEAR/P/RALLEAKTKELVEPTSKEAEEAR
QILAEQAA.

6. The method of claim 2, wherein the modified Toxoplasma cyst peptide comprises an amino acid sequence of
(SEQ ID NO: 1)
EKQLRRVEPEHEDNTRVEAR/P/RALLEAKTKELVEPTSKEAEEAR
QILAEQAA.

7. The method of claim 2, wherein the modified Toxoplasma cyst peptide comprises an amino acid sequence having at least a 50% sequence identity to
(SEQ ID NO: 2)
YENSEDVAVPSDSASTP/DCEEQQEQGDTTLSDHDFHSGGTEQEGL
PETEVAHQHETEEQ.

8. The method of claim 2, wherein the modified Toxoplasma cyst peptide comprises an amino acid sequence having at least a 75% sequence identity to
(SEQ ID NO: 2)
YENSEDVAVPSDSASTP/DCEEQQEQGDTTLSDHDFHSGGTEQEGL
PETEVAHQHETEEQ.

9. The method of claim 2, wherein the modified Toxoplasma cyst peptide comprises an amino acid sequence having at least a 95% sequence identity to
(SEQ ID NO: 2)
YENSEDVAVPSDSASTP/DCEEQQEQGDTTLSDHDFHSGGTEQEGL
PETEVAHQHETEEQ.

10. The method of claim 2, wherein the modified Toxoplasma cyst peptide comprises an amino acid sequence of
(SEQ ID NO: 2)
YENSEDVAVPSDSASTP/DCEEQQEQGDTTLSDHDFHSGGTEQEGL
PETEVAHQHETEEQ.

11. The method of claim 1, wherein the Toxoplasma-associated disease is toxoplasmic encephalitis, toxoplasmic retinitis or Toxoplasma-associated neurological disorders comprising neuropsychiatric and/or degenerative diseases.

12. The method of claim 1, wherein the Toxoplasma-associated disease is toxoplasmic retinitis.

13. The method of claim 1, wherein the subject is treated with an anti-Toxoplasma agent, antibiotics or other therapeutic agents.

14. The method of claim 1, wherein the subject is immunocompromised.

15. A kit comprising the modified Toxoplasma cyst peptides of claim 2.

16-25. (canceled)

26. A synthetic peptide wherein the synthetic peptide comprises an amino acid sequence having at least a 50% sequence identity to
(SEQ ID NO: 1)
EKQLRRVEPEHEDNTRVEAR/P/RALLEAKTKELVEPTSKEAEEAR
QILAEQAA.

27. The synthetic peptide of claim 26, wherein the peptide comprises an amino acid sequence having at least a 75% sequence identity to
(SEQ ID NO: 1)
EKQLRRVEPEHEDNTRVEAR/P/RALLEAKTKELVEPTSKEAEEAR
QILAEQAA.

28. The synthetic peptide of claim 26, wherein the peptide comprises an amino acid sequence having at least an 80%, 85%, 90%, 95%, 98% or 99% sequence identity to
(SEQ ID NO: 1)
EKQLRRVEPEHEDNTRVEAR/P/RALLEAKTKELVEPTSKEAEEAR
QILAEQAA.

29. The synthetic peptide of claim 26, wherein the peptide comprises an amino acid sequence of
(SEQ ID NO: 1)
EKQLRRVEPEHEDNTRVEAR/P/RALLEAKTKELVEPTSKEAEEAR
QILAEQAA.

30-31. (canceled)

32. The synthetic peptide of claim 30, wherein the peptide comprises an amino acid sequence having at least an 80%, 85%, 90%, 95%, 98% or 99% sequence identity to
(SEQ ID NO: 2)
YENSEDVAVPSDSASTP/DCEEQQEQGDTTLSDHDFHSGGTEQEGL
PETEVAHQHETEEQ.

33. (canceled)
